(19) United States
(12) Patent Application Publication
(10) Pub. No.: US 2005/0158747 A1
(43) Pub. Date: Jul. 21, 2005

(54) CFTR modifier genes and expressed polypeptides useful in treating cystic fibrosis and methods and products for detecting and/or identifying same

(76) Inventors: Jeffrey Allen Whitsett, Cincinnati, OH (US); Bruce Jefferson Aronow, Cincinnati, OH (US); Jean Cantwell Clark, Cincinnati, OH (US)

Correspondence Address: HASSE GUTTAG & NESBITT LLC, 7550 CENTRAL PARK BLVD.,, MASON, OH, 45040, US

(21) Appl. No.: 10/999,587

(22) Filed: Nov. 30, 2004

(30) Foreign Application Priority Data
May 30, 2003 (WO) PCT/US03/16896

(51) Int. Cl. C12Q 1/68; A01K 67/00; C07H 21/04; C07K 14/705; A61K 31/4439; A61K 31/353

(52) U.S. Cl. 435/6; 435/69.1; 435/320.1; 435/325; 514/44; 514/338; 530/350; 536/23.5; 800/8; 514/454

(57) ABSTRACT

The discovery that CFTR modifier genes, in particular the Kir4.2 gene, the expressed polypeptide(s), as well as genetic and polypeptide regulators thereof, can be used to treat cystic fibrosis (CF), or at least the conditions that cause CF. Methods and products for detecting and/or identifying CFTR modifier genes, their respective expressed polypeptides, the genetic regulators of such CFTR modifier genes, and the regulators of their respective expressed polypeptides are disclosed. Also disclosed are compositions and methods using these CFTR modifier genes, their respective expressed polypeptides, genetic regulators of these CFTR modifier genes, and/or CFTR modifier polypeptide regulators for the purpose of treating CF, or at least the conditions that cause CF, are disclosed.

### TECHNICAL FIELD

The present application relates to CFTR modifier genes, as well as their expressed polypeptide(s), that are useful in treating cystic fibrosis (CF), or at least the conditions that cause CF. The present application also relates to the use of genetic regulators that modulate such CFTR modifier genes, as well as the use of polypeptide regulators to influence the function and/or activity of the respective expressed polypeptides. The present application further relates to methods and products for detecting and/or identifying such CFTR modifier genes, the respective expressed polypeptides, the genetic regulators that modulate the expression of such modifier genes, and the polypeptide regulators that influence the function and/or activity of the respective expressed polypeptides.

### BACKGROUND OF THE INVENTION

Cystic fibrosis (CF) is the most common fatal genetic disease in humans. See Boat et al, The Metabolic Basis of Inherited Diseases (Scriver, C. R. et al eds., McGraw-Hill, New York (1989)). At the present time, there are many thousands of CF patients in the United States. Despite current standard therapy, the median age of survival is only 26 years. Disease of the pulmonary airways is the major cause of morbidity and is responsible for 95% of the mortality. While many organs are affected in CF, morbidity and mortality in the disease is primarily related to mucus accumulation, recurrent infections and excessive inflammation in the lung.

The protein product of the CF associated gene is called the cystic fibrosis transmembrane conductance regulator (CFTR). See Riordan et al, Science (1989) 245:1066-1073. CFTR is a protein of approximately 1480 amino acids made up of two repeated elements, each comprising six transmembrane segments and a nucleotide binding domain. The two repeats are separated by a large, polar, so-called R-domain containing multiple potential phosphorylation sites. Based on its predicted domain structure, CFTR is a member of a class of related proteins which includes the multi-drug resistance (MDR) or P-glycoprotein, bovine adenyl cyclase, the yeast STE6 protein, as well as several bacterial amino acid transport proteins. See Riordan et al, Science (1989) 245:1066-1073; Hyde et al, Nature (1990) 346:362-365. Proteins in this group, characteristically, are involved in pumping molecules into or out of cells.

CFTR has been postulated to regulate the outward flow of anions from epithelial cells in response to phosphorylation by cyclic AMP (cAMP)-dependent protein kinase or protein kinase C. See Riordan et al, Science (1989) 245:1066-1073; Frizzell et al, Science (1986) 233:558-560; Welsh et al, Nature (1986) 322:467; Li et al, Nature (1988) 331:358-360; Hwang et al, Science (1989) 244:1351-1353. While the pathogenesis of CF is not fully understood, abnormalities in cyclic AMP dependent chloride secretion and excessive sodium reuptake by epithelial cells associated with CFTR deficiency are thought to alter fluid homeostasis at the airway surface liquid (ASL) leading to its dehydration, impaired mucociliary clearance and infection. See Tarran et al, Molecular Cell, (2001) 8:149-58. Since the elucidation of the primary structure of CFTR, a myriad of functions and numerous interactions with other cellular proteins have been ascribed to CFTR. Thus, in addition to the role of CFTR in the regulation of cAMP-dependent chloride transport, this protein may play pleiotropic roles in many cellular processes by interacting with the cytoskeleton, membrane transport proteins, as well as receptors, protein routing and degradation machinery. See Welsh et al, “Cystic Fibrosis,” Metabolic and Molecular Bases of Inherited Disease (8^{th} Ed. 2001), pp. 5121-88.

A number of studies also support the concept that the excessive inflammatory responses occur in the CF lung, but the mechanisms underlying these abnormalities have not been clarified. Changes in levels of IL-8 and other proteins mediating inflammatory signaling including NFκB and iNOS have been associated with CF, in the presence or absence of infection, raising the possibility that abnormalities in CFTR may constitutively alter pathways mediating inflammation. See Khan et al, Am J. Respir. Crit. Care Med., (1995) 151:1075-82; DiMango et al, J. Clin. Invest., (1998) 101:2598-2605; Elmer et al, Am. J. Physiol., (1999) 276:L466-73.

Sequence analysis of the CFTR gene of CF chromosomes has revealed a variety of disease causing mutations. See Cutting et al, Nature (1990) 346:366-369; Dean et al, Cell (1990) 61:863:870; and Kerem et al, Science (1989) 245:1073-1080; Kerem et al, Proc. Natl. Acad. Sci. USA (1990) 87:8447-8451. Indeed, more than 800 distinct mutations in the CFTR gene have been associated with clinical disease characteristic of CF. See Welsh et al, “Cystic Fibrosis,” Metabolic and Molecular Bases of Inherited Disease (8^{th} Ed. 2001), pp. 5121-88. Population studies have indicated that the most common CF mutation, a deletion of the 3 nucleotides that encode phenylalanine at position 508 of the CFTR amino acid sequence (ΔF508), is associated with approximately 70% of the cases of cystic fibrosis. This mutation results in the failure of an epithelial cell chloride channel to respond to cAMP. See Frizzell et al, Science (1986) 233:558-560; Welsh Science (1986) 232:1648-1650.; Li et al, Nature (1988) 331:358-360; Quinton, Clin. Chem. (1989) 35:726-730. In airway cells, this leads to an imbalance in ion and fluid transport. It is widely believed that this causes abnormal mucus secretion, and ultimately results in pulmonary infection and epithelial cell damage.

In the lung, CFTR is distributed primarily in apical regions of airway and submucosal gland epithelial cells. See Engelhardt et al, J. Clin. Invest., (1994) 93:737-49. Abundance and cellular sites of expression of CFTR are strongly influenced by developmental, spatial, and humoral factors, supporting the concept that the expression and function of CFTR are regulated at both transcriptional and post-transcriptional levels. In spite of extensive study, the precise role of CFTR in the pathogenesis of CF disease remains poorly understood. At the clinical level, severity of CF disease is highly variable even among individuals bearing identical mutations, supporting the concept that environmental and hereditary factors can influence the severity of the disorder. See Welsh et al, “Cystic Fibrosis,” Metabolic and Molecular Bases of Inherited Disease (8^{th} Ed. 2001), pp. 5121-88. These clinical observations, and observations demonstrating strain differences in the severity of CF phenotype after CFTR gene targeting or mutation in mice (see Rozmahel et al, Nat. Genet., (1996), 12:280-87), support the concept that the expression of CFTR and its function in cellular processes can be influenced by many genes or pathways intensifying or mollifying CF disease in various organs.

Based on existing knowledge of the CF gene, three general corrective approaches (as opposed to therapies aimed at ameliorating the symptoms) are currently being pursued to reverse the abnormally decreased chloride (Cl⁻) secretion and increased sodium (Na⁺) absorption in CF airways. Defective electrolyte transport by airway epithelia is thought to alter the composition of the respiratory secretions and mucus. See Boat et al, The Metabolic Basis of Inherited Diseases (Scriver, C. R. et al eds., McGraw-Hill, New York (1989)); Quinton, FASEB J. (1990) 4:2709-2717. Hence, pharmacological treatments aimed at correcting the abnormalities in electrolyte transport are being pursued. Trials are in progress with aerosolized versions of the drug amiloride, a diuretic that inhibits sodium (Na⁺) channels, thereby inhibiting sodium absorption. Initial results indicate that the drug is safe and suggest a slight change in the rate of disease progression, as measured by lung function tests. See Knowles et al, N. Eng. J. Med (1990) 322:1189-1194; App, Am. Rev. Respir. Dis. (1990) 141-605. Nucleotides, such as ATP or UTP, stimulate purinergic receptors in the airway epithelium. As a result, they open a class of chloride (Cl⁻) channel that is different from CFTR chloride (Cl⁻) channels. In vitro studies indicate that ATP and UTP can stimulate chloride (Cl⁻) secretion. See Knowles et al, N. Eng. J. Med. (1991) 325-533. Preliminary trials to test the ability of nucleotides to stimulate secretion in vivo, and thereby correct the electrolyte transport abnormalities are underway.

As with all pharmacological agents, issues such as drug toxicity and dosing are important in developing an appropriate pharmacological agent for treating CF. A more fundamental consideration with pharmacological approaches to CF therapy is whether the chloride (Cl⁻) channel activity associated with CFTR is the crucial property that leads to the disease state. There has been speculation that there is another as yet unidentified component of the CFTR system that is the key regulator, and that if this were the case, it would be possible that a pharmacological approach based on chloride (Cl⁻) transport might successfully adjust ion balance, but still not relieve the fundamental physiological problem. See U.S. Pat. No. 5,639,661 (Welsh et al), issued Jun. 17, 1997. If this were the case, it is possible that a pharmacological approach based on chloride (Cl⁻) transport might successfully adjust ion balance, but still not relieve the fundamental physiological problem.

A second approach to treating cystic fibrosis, “protein replacement,” seeks to deliver functional, recombinant CFTR to CF mutant cells to directly augment the missing CFTR activity. The concept of protein replacement therapy for CF is simple: a preparation of highly purified recombinant CFTR formulated in some fusogenic liposome or reassembled virus carrier delivered to the airways by instillation or aerosol. However, attempts at formulating a CF protein replacement therapeutic have met with difficulties. For example, CFTR is not a soluble protein of the type that has been used for previous protein replacement therapies or for other therapeutic uses. There may also be a limit to the amount of a membrane protein with biochemical activity that can be expressed in a recombinant cell. For example, there are reports in the literature of 10⁵-10⁶ molecules/cell representing the upper limit (see Wang et al, J. Biol. Chem (1989) 264:14424), compared to 2000 molecules/second/cell being reported for secreted proteins such as EPO, insulin, growth hormone, and tPA.

In addition to limited expression capabilities, the purification of CFTR, a membrane bound protein, is more difficult than purification of a soluble protein. Membrane proteins require solubilization in detergents. However, purification of CFTR in the presence of detergents represents a less efficient process than the purification process required of soluble proteins. Other potential obstacles to a protein replacement approach include: (1) the inaccessibility of airway epithelium caused by mucus build-up and the hostile nature of the environment in CF airways; (2) potential immunogenicity; and (3) the fusion of CFTR with recipient cells can be inefficient.

A third approach to cystic fibrosis treatment is a gene therapy approach in which DNA encoding CFTR is transferred to CF defective cells (e.g., of the respiratory tract). However, methods to introduce DNA into cells are generally inefficient. Since viruses have evolved very efficient means to introduce their nucleic acid into cells, many approaches to gene therapy make use of engineered defective viruses. However, viral vectors have limited space for accommodating foreign genes. For example, adeno-associated virus (AAV) although an attractive gene therapy vector in many respects, has only 4.5 Kb available for exogenous DNA. DNA encoding the full length CFTR gene represents the upper limit. Furthermore, gene therapy approaches to CF will face many of the same clinical challenges as protein therapy.

Although there has been notable progress in developing therapies for treating CF based on current knowledge of the gene encoding CFTR, the expressed protein product and mechanism of action, there are still obstacles confronting every approach to treating CF. Morbidity and mortality in patients with CF is strongly associated with pulmonary disease caused by mucous accumulation, inflammation and infection. However, deletion of CFTR in mice does not cause significant pulmonary disease, suggesting that expression of alternative channels or other complementary genes maintain pulmonary homeostasis in the mouse. Indeed, lung disease in CF mouse models has been found to be highly variable. In general, pulmonary disorder can be mild or absent in CFTR mutant mice. This suggests that other genes expressed in the lung may strongly influence the function of CFTR.

While numerous in vitro and in vivo models have been developed for the study of CFTR, analysis of genomic responses to the presence or absence of CFTR are complicated by heterogeneity of cell models, and culture conditions that can influence cell function and gene expression independently of CFTR. Direct RNA analysis of pulmonary tissue from humans with CF is complicated by the nearly ubiquitous, severe pulmonary infections that can secondarily modify cellular responses and gene expression, complicating identification of responses to CFTR in vivo.

Accordingly, there is still a need to discover and identify genes or agents that can modify or enhance ion transport, chloride (Cl⁻) transport or other co-transported ions, as well as other activities that CFTR influences. This would provide the ability to define therapeutic targets for CF, as well as potentially new genes, agents and therapies for treating CF. In particular, it would be desirable to discover or identify alternative pathways for influencing ion transport, in particular chloride (Cl⁻) transport, that could be more effective in treating CF.

### BRIEF DESCRIPTION OF THE INVENTION

The present invention relates to the discovery that CFTR modifier genes, and in particular the Kir4.2 gene, as well as their expressed polypeptide(s), can be useful in treating the disease of cystic fibrosis (CF) by providing a compensatory function and/or activity for CF-affected cells that either lack the ability to express CFTR, or express a mutated CFTR that provides ineffective or less effective CFTR function and/or activity. The present invention further relates to the discovery of genetic regulators that can modulate the expression of such CFTR modifier genes, as well as polypeptide regulators that influence the function and/or activity of the respective expressed polypeptides.

Accordingly, the present invention provides methods and products for detecting and/or identifying CFTR modifier genes, the respective expressed CFTR modifier polypeptides, the genetic regulators that modulate the expression of such CFTR modifier genes, and the regulators that influence the function and/or activity of the respective expressed CFTR modifier polypeptides. Some embodiments of the detection/identification methods and products of the present invention include: (1) the use of the Kir4.2 gene and other CFTR modifier genes, as well as their expressed polypeptides, as screens or assays for potential agents (e.g., drugs) that can modulate gene expression (i.e., gene regulators), or can influence the functional properties of the expressed polypeptides (i.e., polypeptide regulators); (2) the use of CFTR-deficient transgenic nonhuman mammals (e.g., mice) to identify potential CFTR modifier genes, as well as their respective expressed CFTR modifier polypeptides; and (3) introducing suitable vectors comprising CFTR modifier genes and/or gene promoters into mammalian (human and nonhuman), yeast, or insect cells or cell lines so that changes in gene expression or polypeptide function and/or activity can be used in an assay system to detect and/or identify potential genetic and/or polypeptide regulators.

The present invention also provides compositions and methods using these CFTR modifier genes, their respective expressed polypeptide(s), as well as other agents, for the purpose of treating CF, including: (1) the use of genetic regulators to modulate CFTR modifier gene expression in CF-affected cells; (2) the use of polypeptide regulators to influence (e.g., enhance) the function and/or activity of CFTR modifier polypeptide(s) in CF-affected cells; (3) the delivery of CFTR modifier polypeptides to CF-affected cells; and/or (4) the delivery of CFTR modifier genes to CF-affected cells, with or without other treatments or agents.

It has been surprisingly found that Kir4.2, as well as other CFTR modifier genes, is unexpectedly able to compensate for the absence of CFTR, or the presence of less effective or ineffective mutant CFTRs (such as CFTR ΔF508) so as to treat CF, or at least the conditions that cause CF. In particular, it has been found that the Kir4.2 gene unexpectedly influences and potentiates chloride (Cl⁻) ion transportation by providing potassium (K⁺) channel(s) as an alternative pathway(s). In addition, it has been surprisingly found that the expressed polypeptide(s) of the Kir4.2 gene that provide these potassium (K⁺) channel(s) can be activated and/or regulated in response to various agents, such as cyclic AMP (cAMP) stimulating agents (e.g., forskolin and IBMX) that stimulate chloride (CI⁻) ion transportation via CFTR-dependent channels. Accordingly, because Kir4.2 activation also potentiates chloride (Cl⁻) ion transport, pharmacological agents that modulate the expression of Kir4.2 (transcriptionally or post-transcriptionally), or influence the function and/or activity of the expressed Kir4.2 polypeptide(s), can also beneficially influence potassium (K⁺) ion transport, and thus chloride (Cl⁻) ion transport. Indeed, an advantage of the present invention is the ability to treat CF by modulating the expression of endogenous CFTR modifier genes and/or influencing the function/activity of endogenous CFTR modifier polypeptides that can beneficially impact ion transport by alternative pathways (e.g., in the case of Kir4.2, by potassium (K⁺) channel(s)).

### BRIEF DESCRIPTION OF DRAWINGS

FIG. 1 is a graph of the differential expression patterns (relative intensity plotted on y-axis v. pairs of mice of increasing age on x-axis) of mRNAs harvested from the lungs of FABP-hCFTR/mCFTR(−/−) CFTR-deficient gut-corrected mice, versus wild type CFTR (+/+) mice with filtering (p-value<0.05) showing 27 genes, including the Kir4.2 gene, that are potentially CFTR modifier genes.

FIG. 2 is a bar graph showing the increase in Kir4.2 mRNA in CFTR deficient gut-corrected, versus wild-type control, mouse lung.

FIG. 3 is a graph showing the activity (pA/pF on y-axis v. Vₘ in mV on the x-axis) of cloned potassium (K⁺) channel(s) expressed by the Kir4.2 gene in response to cAMP stimulating agents (combination of forskolin and IBMX) versus control.

FIG. 4 shows in the left panel a histogram of log ratio and gene frequency, and in the right panel an outlier box plot, of the distribution of various RNAs from CFTR (−/−) and CFTR (+/+) mice, where the ends of the dashed lines, denoted by an x and y markers, are the outliers identified from their respective quartiles.

FIG. 5 is an image of a two dimensional hierarchial clustering of 315 genes/expressed sequence tags (ESTs) that are significantly altered in response to presence or absence of CFTR.

FIG. 6 is an image of an expression profile chart of 54 selected RNAs that are consistently altered in response to the absence of CFTR.

FIG. 7 is an image of the respective hierarchical clustering of the 54 selected RNAs of FIG. 6.

FIGS. 8, 9, 10 and 11 represent, respectively, bar graphs showing the real time PCR analysis of the mRNAs for Grind2d (FIG. 8), Kir4.2 (FIG. 9), CEBPδ (FIG. 10) and TNFAIP3 (FIG. 11).

FIGS. 12, 13, 14 and 15, are images, respectively, of lung tissue obtained after fixation from iFABP-hCFTR, CFTR (−/−) mice (FIGS. 13, 15) and iFABP-hCFTR, CFTR (+/+) littermates (FIGS. 12, 14) at three months of age

### BRIEF DESCRIPTION OF SEQUENCE LISTINGS

SEQ ID NO:1 shows the nucleotide sequence of the cDNA of the mouse Kir4.2 gene.

SEQ ID NO:2 shows the Kir4.2 polypeptide expressed by the mouse Kir4.2 gene.

SEQ ID NO:3 shows the nucleotide sequence of the cDNA of the human Kir4.2 gene.

SEQ ID NO:4 shows a variant of the nucleotide sequence of the cDNA of the human Kir4.2 gene.

SEQ ID NO:5 shows another variant of the nucleotide sequence of the cDNA of the human Kir4.2 gene.

SEQ ID NO:6 shows the Kir4.2 polypeptide expressed by the human Kir4.2 gene of SEQ ID NOs. 3-5.

SEQ ID NO:7 shows another variant of the nucleotide sequence of the cDNA of the human Kir4.2 gene.

SEQ ID NO:8 shows the Kir4.2 polypeptide expressed by the human Kir4.2 gene of SEQ ID NO. 7.

### DETAILED DESCRIPTON OF THE INVENTION

Definitions

As used herein, the term “gene” means a sequence of genetic material (e.g., DNA and RNA) that carries the information encoding a polypeptide (e.g., protein).

As used herein, the term “RNA” can refer interchangeably to RNA, mRNA or tRNA.

Unless otherwise indicated herein, the term “polypeptide” means a protein, polypeptide or peptide.

As used herein, the term “vector” means an agent comprising, consisting essentially of, or consisting of a DNA or RNA capable of introducing a nucleic acid sequence(s) into a cell, resulting in the expression of the nucleic acid sequence(s) in the cell.

As used herein, the term “expression vector” means a modified plasmid or virus that carries a gene or cDNA into a suitable host cell and there directs expression or synthesis of the encoded polypeptide.

As used herein, the terms “plasmid” and “cloning vector” are used interchangeably to mean a circular, typically small extrachromosomal DNA molecule capable of autonomous replication in a cell.

As used herein, the terms “Cystic Fibrosis Transmembrane Conductance Regulator polypeptide” or “CFTR polypeptide” refer to a protein of approximately 1480 amino acids containing two membrane-spanning domains (MSDs), two nucleotide binding domains (NBDs) and a unique R domain, that functions as a chloride (Cl⁻) channel regulated by phosphorylation and by nucleoside triphosphates. The term “CFTR polypeptide” can also refer to those portions of the CFTR gene that retain functional domains of the CFTR gene.

As used herein, the terms “CFTR Δ508” and “CFTR ΔF508” are used interchangeably to refer to the CFTR mutant polypeptide that results from the failure to encode phenylalanine at position 508 of the CFTR amino acid sequence

As used herein, the term “cystic fibrosis transmembrane conductance regulator (CFTR) function or activity” refers to functions normally performed by wild-type CFTR. Such functions can include mediation, regulation or control of ion, (e.g. chloride (Cl⁻) ion) transport across cellular membranes.

As used herein, the term “cystic fibrosis (CF)-defective or affected cell” refers to a cell that lacks cystic fibrosis transmembrane conductance regulator function either due to the absence of CFTR, or due to a CFTR mutant polypeptide that is unable to provide CFTR function and/or activity, or is less effective in providing CFTR function and/or activity. Examples of such cells include CFTR mutants (e.g., CFTR ΔF508) of which 1300 different varieties have been identified to date. See, for example, Kunzelmann et al, “Pharmacotherapy of the Ion Transport Defect in Cystic Fibrosis,” Clin. Exper. Pharm. Phys. (2001) 28:857-67; Welsh et al, “Molecular Mechanisms of CFTR Chloride Channel Dysfunction in Cystic Fibrosis,” Cell (1993) 73:1251-54.

As used herein, the terms “CFTR modifier gene” and “CFTR compensatory gene” are used interchangeably to mean a gene that is capable of compensating for the activity or function of CFTR in a CF-affected cell, including expressing polypeptides that are capable of compensating for the function and/or activity of CFTR in a CF-affected cell. Examples of CFTR modifier genes include the following up-regulated genes: EST Affymetrix ID#92319 (an ubiquitin family member that may mediate protein trafficking); guanine nucleotide binding protein α subunit (a G protein-coupled receptor pathway protein); Repetin (GenBank accession number X99251); membrane glycoprotein (GenBank accession number Z22552); ras-related dexamethasone inducible protein (DEXRAS1) mRNA; SWAP-70 mRNA; vq96e09.41 cDNA; zinc finger protein (Peg3) mRNA; uo89c05.x1 cDNA; and ATP-sensitive inward rectifier potassium channel 14 (GenBank accession number AI314692). Examples of CFTR modifier genes also include the following down-regulated genes: Preproapelin (GenBank accession number AB023494); Caspase-12 (GenBank accession number Y13090); islet cell autoantigen 1 (GenBank accession number U37186); Natriuretic peptide precursor type A (GenBank accession number K02781); mSox7 (GenBank accession number AB023419); secreted frizzled related protein sFRP-2 (Sfrp2) mRNA (GenBank accession number U88567); U[-M-BH1-ang-b-04-0-U].s1 cDNA (GenBank accession number AW050325); C88243 cDNA (GenBank accession number C88243); U[-M-BH0-ajq-h-03-0-U].s1 (GenBank accession number AI853682); IB3 (GenBank accession number X79131); Butyrylcholinesterase mRNA (GenBank accession number M99492); homeo box A5 (GenBank accession number Y00208); Connexin 37 Gap junction membrane channel protein alpha 4 (GenBank accession number X57971); Wnt10a mRNA (GenBank accession number U61969); Calnexin (GenBank accession number L18888); Cystic fibrosis transmembrane conductance regulator homologue (GenBank accession number M60493); and mRNA similar to human hematopoietic specific protein 1 (GenBank accession number X84797). Genes that compensate for the CF disease state in the presence of ineffective and/or less effective CFTR mutants are also referred to herein as CFTR modifier genes.

The following is a table of other genes that have been found to be up-regulated in mCFTR (−/−) mice versus mCFTR (+/+) mice:

TABLE 1
UP-Regulated Genes

| Gene | Symbol | Ratio | P-value | Category | Accession No. |
|---|---|---|---|---|---|
| envelope protein (env) gene, 3 end | Env | 4.02 | 1.19E−03 | antigen | M90535 |
| X-linked lymphocyte-regulated 3b | Xlr3b | 2.25 | 1.48E−02 | antigen | NM_011727 |
| polymyositis/scleroderma autoantigen 2 | Pmsc12 | 2.53 | 2.59E−02 | antigen | NM_016699 |
| calbindin-D9K | Calb3 | 1.77 | 3.33E−02 | calcium binding | NM_009789 |
| phosphoglycerate mutase 2 | Pgam2 | 2.09 | 9.10E−04 | glycolysis | NM_018870 |
| | | | | metabolism | |
| natriuretic peptide receptor 3 | Npr3 | 2.37 | 4.85E−03 | inflammatory | NM_008728 |
| | | | | response | |
| chitinase, acidic | Chia- | 1.50 | 8.75E−03 | inflammatory | NM_023186 |
| | pending | | | response | |
| colony stimulating factor 3 receptor | Csf3r | 1.84 | 2.86E−02 | inflammatory | NM_007782 |
| (granulocyte) | | | | response | |
| tumor necrosis factor, alpha-induced | Tnfaip3 | 5.07 | 2.96E−02 | inflammatory | NM_009397 |
| protein 3 | | | | response | |
| Mus musculus interleukin 4(Il-4) | Il4 | 1.32 | 3.85E−02 | inflammatory | U01310 |
| mRNA, complete cds. | | | | response | |
| interleukin 1 beta | Il1b | 1.67 | 5.38E−02 | inflammatory | BC013644 |
| | | | | response | |
| S100 calcium binding protein A8 | S100a8 | 1.92 | 9.48E−03 | inflammatory | NM_013650 |
| (calgranulin A) | | | | response/calcium | |
| | | | | binding | |
| S100 calcium binding protein A9 | S100a9 | 2.39 | 1.93E−02 | inflammatory | NM_009114 |
| (calgranulin B) | | | | response/calcium | |
| | | | | binding | |
| potassium inwardly-rectifying | Kcnj15 | 3.28 | 1.59E−02 | ion transport | NM_019664 |
| channel, subfamily J, member 15 | | | | | |
| putative adapter protein binds | Mig- | 2.12 | 2.16E−02 | RHO GTPase | NM_133753 |
| monomeric GTPases of the Rho | 6/Gene33 | | | activator/inflammatory | |
| subfamily | | | | response | |
| paternally expressed 3 | Peg3 | 1.74 | 3.22E−02 | signal transduction | AB003040 |
| secretory granule neuroendocrine | Sgne1 | 6.76 | 4.33E−02 | signal transduction | NM_009162 |
| protein 1, 7B2 protein | | | | | |
| claudin 8 | Cldn8 | 2.86 | 4.16E−02 | tight | BC003868 |
| | | | | junction/transport | |
| Mouse c-fos oncogene. | c-Fos | 1.75 | 3.87E−03 | transcription | V00727 |
| | | | | regulation | |
| period homolog (Drosophila) | Per | 1.76 | 1.90E−02 | transcription | NM_011065 |
| | | | | regulation | |
| Kruppel-like factor 1 (erythroid) | Klf1 | 2.63 | 3.84E−02 | transcription | NM_010635 |
| | | | | regulation | |
| RARG-1: retinoic acid repressible | RARG-1 | 1.50 | 3.86E−02 | transcription | NM_023554 |
| protein | | | | regulation | |
| CCAAT/enhancer binding protein | Cebpd | 1.61 | 4.15E−02 | transcription | NM_007679 |
| (C/EBP), delta | | | | regulation | |
| solute carrier family 38, member 4 | Slc38a4 | 2.03 | 1.95E−04 | transport | NM_027052 |
| proteasome (prosome, macropain) | Psmc3 | 1.54 | 2.45E−02 | transport | NM_008948 |
| 26S subunit, ATPase 3 | | | | | |
| EST, express in lung | | 3.92 | 2.98E−02 | unknown | BG869733 |
| Glutamate receptor, ionotropic, | Grin2d | 3.50 | 4.46E−02 | Transport/receptor | NM_008172 |
| NMDA2D (epsilon 4) | | | | | |
| M. musculus membrane glycoprotein | | 1.62 | 5.73E−03 | unknown | Z22552 |
| gene. | | | | | |
| RIKEN cDNA 1100001G20 | 100001G20 | 1.56 | 1.74E−02 | unknown | AV006463 |
| | Rik | | | | |

The following is a table of other genes that have been found to be down-regulated in mCFTR (−/−) mice versus mCFTR (+/+) mice:

TABLE 2
Down-Regulated Genes

| Gene | Symbol | Ratio | P-value | Category | Accession No. |
|---|---|---|---|---|---|
| SA rat hypertension-associated | Sah | −4.44 | 8.69E−04 | blood pressure | NM_016870 |
| homolog | | | | regulation | |
| adenylate cyclase 4 | Adcy4 | −1.34 | 3.72E−02 | cAMP biosynthesis | NM_080435 |
| formin-like | Fmnl | −2.21 | 1.04E−03 | cell growth | NM_019679 |
| insulin-like growth factor binding | Igfbp2 | −1.23 | 6.99E−04 | cell growth | NM_008342 |
| protein 2 | | | | | |
| insulin-like growth factor binding | Igfbp7 | −1.38 | 4.23E−02 | cell growth | NM_008048 |
| protein 7 | | | | | |
| procollagen, type XV | Col15a1 | −2.07 | 4.30E−02 | collagen | NM_009928 |
| RAD51-like 1 (S. cerevisiae) | Rad5111 | −1.56 | 1.32E−02 | DNA | NM_009014 |
| | | | | recombination/cell | |
| | | | | growth | |
| neurofilament, heavy polypeptide | Nfh | −1.92 | 4.69E−05 | intermediate filament | M35131 |
| adaptor protein complex AP-2, alpha | Ap2a1 | −1.52 | 3.72E−02 | intracellular protein | NM_007458 |
| 1 subunit | | | | traffic | |
| kinesin family member 3a | Kif3a | −1.48 | 3.72E−02 | intracellular protein | NM_008443 |
| | | | | traffic | |
| ADP-ribosylation factor 5 | Arf5 | −1.34 | 1.06E−02 | intracellular protein | NM_007480 |
| | | | | traffic | |
| lipase, hormone sensitive | Lipe | −2.34 | 1.82E−02 | Lipid catabolism/cell | NM-010719 |
| | | | | growth | |
| Ki antigen; Psme3 gene for PA28 | Psme3 | −1.57 | 5.44E−06 | Protein degradation | NM_011192 |
| gamma subunit | | | | | |
| metallocarboxypeptidase 1 | CPX-1 | −3.60 | 4.44E−03 | Protein degradation | NM _019696 |
| yolk sac gene 2 | Ysg2 | −3.21 | 1.38E−02 | Protein degradation | NM_011734 |
| parathyroid hormone precursor (Pth) | Pth | −2.13 | 2.68E−03 | signal transduction | NM_020623 |
| gene | | | | | |
| tryptophan 2,3-dioxygenase | Tdo2 | −2.01 | 2.15E−02 | signal transduction | NM _019911 |
| beta-3-adrenergic receptor | Adrb-3 | −2.01 | 1.31E−02 | signal transduction | NM_013462 |
| Janus kinase 3 | Jak3 | −1.49 | 1.24E−02 | signal transduction | NM_010589 |
| nuclear receptor subfamily 2, group | Nr2f1 | −1.46 | 3.28E−03 | signal transduction | NM_010151 |
| F, member 1 | | | | | |
| interferon regulatory factor 1 | Irf1 | −1.19 | 8.51E−03 | transcription | NM_008390 |
| | | | | regulation | |
| cystic fibrosis transmembrane | Cftr | −2.80 | 3.63E−07 | transport | NM_021050 |
| conductance regulator homolog | | | | | |
| Mouse gap junction gene connexin | GJA4 | −1.81 | 2.12E−02 | transport/cell-cell | NM_008120 |
| 37. | | | | communication | |
| DNA segment, Chr 4, ERATO Doi 13 | D4Ertd13e | −2.07 | 4.45E−02 | unknown | AI842362 |
| Riken cDNA 2700022J23 gene | 2700022j23 | −1.45 | 3.72E−02 | unknown | AI853682 |
| | Rik | | | | |

As used herein, the terms “treating cystic fibrosis (CF),” “cystic fibrosis (CF) treatments” and like phrases include treatments that provide any detectable reduction, alleviation, amelioration, benefit or other positive effect for CF-affected cells (directly or indirectly), as well as any treatment that provides any detectable reduction, alleviation, amelioration, benefit or other positive improvement with regard to any condition that causes or is associated with CF, including deficiencies in ion transport into and out of the CF-affected cell.

As used herein, the phrase “CF modifier gene therapy” refers to the transfer of genetic material (e.g., DNA or RNA) encoding compensatory CFTR function and/or activity into a CF-affected cell to reduce, alleviate, or ameliorate the conditions of, or otherwise positively treat, cystic fibrosis (CF).

As used herein, the term “CFTR(+/+)” refers to the wild-type CFTR mice.

As used herein, the term “CFTR(−/−)” refers to the CFTR deficient mice.

As used herein, the term “FABP-hCFTR” refers to human CFTR that is expressed in the gut of transgenic mice.

As used herein, the term “FABP-hCFTR/mCFTR(−/−)” refers to CFTR deficient gut-corrected transgenic mice.

As used herein, the term “CHO” refers to Chinese Hamster Ovary.

As used herein, the term “cAMP” refers to cyclic adenosine monophosphate.

As used herein, the term “SP-C” refers to Surfactant Protein-C, a major component of the pulmonary surfactant fluid that lines the airways of mammals and other air-breathing animals.

As used herein, the term “SPC-hΔ508” refers to transgenic mice expressing human CFTR Δ508 in pulmonary epithelial cells under the regulation of SP-C promoter sequences.

As used herein, the term “Kir4.2 gene” refers to the inward rectifying potassium (K⁺) channel gene that expresses a polypeptide(s) that functions as a potassium (K⁺) channel(s) and has previously been found to be expressed in the kidney and lung during development and in several adult tissues, including kidney and brain. The Kir4.2 gene is also referred to as KIR4.2 or KCNJ15, and is localized on chromosome 21 in humans. See Gosset et al, “A New Inward Rectifier Potassium Channel Gene (KCNJ15) Localized on Chromosome 21 in the Down Syndrome Chromosome Region 1 (DCR 1),” Genomics (1997) 44:237-41, which is incorporated by reference. As with many genes, allelic variants can produce essentially similar polypeptides, and examples from humans are provided herein. The cDNA of the mouse Kir4.2 gene has the nucleotide sequence shown in SEQ ID NO:1 (GenBank accession number AF 085696), while the cDNA of the human Kir4.2 gene has the nucleotide sequence shown in SEQ ID NO:3 (GenBank accession number NM_{—}170737), SEQ ID NO:4 (GenBank accession number NM_{—}170736), SEQ ID NO:5 (GenBank accession number NM_{—}002243) or SEQ ID NO:7 (GenBank accession number Y10745). The Kir4.2 gene has increased levels of expression in the absence of normal CFTR, and in the presence of CFTR Δ508 combined with the absence of normal CFTR. It has been found that Kir4.2 can be expressed in the relevant regions of the pulmonary airway epithelium, thus increasing expression of the respective Kir4.2 polypeptide (shown in SEQ ID NO:2 for mouse, and in SEQ ID NOs: 6 and 8 for human) or enhancing the activity of this polypeptide to bypass CFTR-dependent defects in chloride (Cl⁻) transport and cell function. Analysis of the regulatory regions of the Kir4.2 and CFTR genes show many similarities, linking their potential function and validating the unexpected discovery that Kir4.2 is a CFTR modifier gene. See Durell et al, “A Family of Putative Kir Potassium Channels in Prokaryotes,” BMC Evolutionary Biology (2001), pp. 1-9; Fakler et al, “Heterooligomeric Assembly of Inward-Rectifier K+ Channels from Subunits of Different Subfamilies: Kir2.1 (IRK1) and Kir 4.1 (BIR10),” Pflugers Arch-Eur. J. Physiol. (1996) 433:77-83 (herein incorporated by reference) for other gene family members that are related to the Kir4.2 gene.

As used herein, the term “gene promoter” refers to that portion of the nucleotide sequence of the gene that regulates, controls or otherwise modulates (e.g., stimulates or suppresses) the expression by the particular gene. For example, a gene promoter can enhance transcription and/or translation of the gene, thus increasing the mRNA levels transcribed from that gene.

As used herein, the terms “gene expression” and “gene transcription,” are used interchangeably to refer to initial steps at the level of the DNA molecule that lead to the production of a gene polypeptide product. Thus, gene transcription is to be understood herein as culminating in gene expression, or the production of the polypeptide encoded by the gene so transcribed or expressed.

As used herein, the term “reporter gene” refers to genetic material, usually DNA, introduced into a cell where the reporter gene is expressed under favorable conditions, so as to indicate that the criteria for achieving those favorable conditions have been achieved. These favorable conditions can include, but are not limited to, pH, ion flow, and the presence of an appropriate repertoire of transcription factors, or other intracellular molecules or factors.

As used herein, the term “transcription factors” refers to a wide array of intracellular polypeptides that bind DNA or cause other polypeptides to bind DNA before, during, or after the process of gene transcription.

As used herein, the term “genetic regulator” means an agent that modulates the expression of a gene. In the present invention, genetic regulators usually refer to an agent that increases or decreases the intracellular level of at least one cellular polypeptide (e.g., protein) expressed by a CFTR modifier gene, particularly in a CF-affected cell. Genetic regulators can include endogenous cellular polypeptides, pharmacological agents or other biologically active molecules.

As used herein, the term “polypeptide regulator” refers to an agent capable of influencing (e.g., increasing or decreasing) the cellular function and/or activity of at least one CFTR modifier polypeptide in a CF-affected cell. Polypeptide regulators can stimulate, enhance, or increase the function and/or activity of the polypeptide, as well as inhibit or decrease such function or activity. As used herein, the function and/or activity of the CFTR modifier polypeptide(s) in a CF-affected cell is influenced by the polypeptide regulator so as to beneficially regulate, control or otherwise modulate ion transport (e.g., chloride (Cl⁻), potassium (K⁺), etc.) or other CFTR function and/or activity, either directly or indirectly, so as to alleviate or otherwise positively treat CF.

As used herein, the term “pharmaceutically acceptable salt” means non-toxic salts of compounds (which are generally prepared by reacting the free acid with a suitable organic or inorganic base) and include, but are not limited to, the acetate, benzenesulfonate, benzoate, bicarbonate, bisulfate, bitartrate, borate, bromide, calcium, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandlate, mesylate, methylbromide, methylnitrate, methylsulfate, mucate, napsylate, nitrate, oleate, oxalate, pamaote, palmitate, panthothenate, phosphate, diphospate, polygalacturonate, salicylate, stearate, subacetate, succinate, tannate, tartrate, teoclate, tosylate, triethiodide, and valerate salts, as well as mixtures of these salts.

As used herein, the terms “agent,” “pharmaceutical,” and “drug” are used interchangeably to refer to a pharmacological composition, formulation or compound, including those useful as a genetic and/or a polypeptide regulator.

As used herein, the term “mammal” refers to humans and nonhuman mammals, including primates (e.g., humans, monkeys, baboons, macaques), dogs, cats, rabbits rats, gerbils, hamsters, mice, horses, cows, goats, and other species commonly known as mammals.

As used herein, the term “subject” is intended to include mammals susceptible to CF. The term “subject” is further intended to include transgenic nonhuman mammals. “Subjects” are also referred to herein interchangeably as “patients.”

As used herein, the term “comprising” means various agents, compositions, compounds, genes, polypeptides, components, steps and the like can be conjointly employed in the present invention. Accordingly, the term “comprising” encompasses the more restrictive terms “consisting essentially of” and “consisting of.”

All amounts, parts, ratios and percentages used herein are by weight unless otherwise specified.

2. Detection and Identification of CFTR Modifier Genes, Genetic Regulators and Polypeptide Regulators

The present invention relates to the discovery that the expression of a number of genes (i.e., CFTR modifier genes such as the Kir4.2 gene) is altered in nonhuman mammals (e.g., mice) so as to provide apparently normal pulmonary function, despite lacking CFTR gene expression, or with expression of a mutated CFTR that is unable or less efficient in providing CFTR function or activity, such as is found in a subject having CF disease. The altered expression of these CFTR modifier genes has been found to be a compensatory adaptation. Since loss of CFTR function and/or activity is normally disease producing in humans and other nonhuman mammals (e.g., mice), the CFTR modifier genes can compensate for the effects of CFTR, especially the loss of CFTR expression. As a result, these compensatory CFTR modifier genes, as well as their expressed polypeptide(s), can restore pulmonary homeostasis and can be useful in treating CF.

Accordingly, the present invention relates to methods and products for detecting and/or identifying CFTR modifier genes, including up-regulated genes such as those listed in Table 1, as well as down-regulated genes such as those listed in Table 2, their expressed CFTR modifier polypeptides, genetic regulators that modulate the expression of such modifier genes, and regulators that influence the function and/or activity of their respective expressed polypeptides. Various biological materials and methods can be used to detect and/or identify potential CFTR modifier genes and their respective expressed polypeptides, potential genetic regulators of CFTR modifier genes, as well as potential polypeptide regulators that influence the function and/or activity of the respective expressed polypeptides, including drug screens; assays; arrays and/or probes of biological molecules (e.g., nucleotides or polypeptides); as well as mouse and other nonhuman mammal models. These detection and identification methods and products typically involve contacting a sample containing the potential CFTR modifier gene, CFTR modifier polypeptide, genetic regulator of a CFTR modifier gene and/or regulator of a CFTR modifier polypeptide, with an indicator that identifies when a potential CFTR modifier gene, CFTR modifier polypeptide, genetic regulator of a CFTR modifier gene and/or regulator of a CFTR modifier polypeptide is present in the sample.

Suitable products, kits, and assays for detecting and/or identifying CFTR modifier genes, CFTR modifier polypeptides, genetic regulators of CFTR modifier genes and regulators of CFTR modifier polypeptides can be in the form of arrays; probes; hybridization assays; sandwich assays; the use of DNA sequencing and identification by hybridization (including using discrete multiple probe analysis); use of sequencing, fingerprinting and mapping of nucleotides and polypeptides; and arrays of polypeptide (e.g., protein)-capture agents. See, for example, U.S. Pat. No. 5,445,934 (Fodor et al), issued Aug. 29, 1995; U.S. Pat. No. 6,027,800 (Cronin et al), issued Feb. 22, 2000; U.S. Pat. No. 6,045,996 (Cronin et al), issued Apr. 4, 2000; U.S. Pat. No. 6,077,673 (Chenchik et al); U.S. Pat. No. 6,268,210 (Baier et al), issued Jul. 31, 2001; U.S. Pat. No. 6,270,961 (Drmanac), issued Aug. 7, 2001; U.S. Pat. No. 6,355,432 (Fodor et al), issued Mar. 12, 2002; and, all of which are incorporated by reference. For example, in the case of an array, a substrate with a surface comprising a plurality (or more typically multiplicity) of biologically active materials are attached to the surface in discrete regions, the biological materials being capable of identifying potential CFTR modifier genes, CFTR modifier polypeptides, genetic regulators of CFTR modifier genes and/or regulators of CFTR modifier polypeptides. This array can then be used to screen for potential CFTR modifier genes, CFTR modifier polypeptides, genetic regulators of CFTR modifier genes and/or regulators of CFTR modifier polypeptides. For example, the array could be contacted with a sample containing the potential CFTR modifier gene, CFTR modifier polypeptide, genetic regulator of CFTR modifier gene and/or regulator of a CFTR modifier polypeptide, the array also having associated therewith an indicator for identifying if a potential CFTR modifier gene, CFTR modifier polypeptide, genetic regulator of CFTR modifier genes and/or regulator of CFTR modifier polypeptide is present in the sample.

a. CFTR Modifier Genes and Polypeptides

Methods for detecting and/or identify CFTR modifier genes typically look for genes that compensate for alterations in CFTR expression or activity. For example, changes in gene expression in nonhuman mammals (e.g., in mice) with transgenic manipulations of CFTR expression can reveal those genes that compensate for loss of (or reduction in) CFTR expression or activity. Methods and products for detecting and/or identifying CFTR modifier genes include the use of transgenic nonhuman mammals as a source of RNA to assay for changes in gene expression in CF disease. These methods and products generally involve contacting a sample containing a mixture of potential CFTR modifier genes, preferably an isolate of mRNA or total cellular RNA, with an indicator that identifies when a potential CFTR modifier mRNA is present in the sample. These methods and products can include but are not limited to RT-PCR, Northern blotting, microarray, Real Time PCR, or gene chip techniques such as Affymetrix. For example, an mRNA sample comprising a mixture of unknown mRNAs can be isolated from transgenic mice with systemic or lung-specific CFTR gene mutations and then assayed for changes in expression of genes that ameliorate the effects of CF disease. The transgenic mice can include CFTR-null mutant mice with transgenic intestine-specific expression of CFTR that allows the CFTR-null mutant mice to survive through adulthood. Also included is the use of CFTR-null mutant mice with lung-specific transgenic expression of the human Δ508-mutated CFTR gene. In methods involving the use of CFTR-deficient transgenic mice to identify CFTR modifier genes that compensate for alterations in CFTR expression or activity, such as those alterations or mutations that manifest in CF disease, the RNA can be harvested from any organ or tissue affected by CF disease, including but not limited to, upper airway epithelium, lung, pancreas, and intestine of the CFTR-deficient mice.

One embodiment of such a detection and/or identification method comprises the steps of: (1) providing a CFTR mutant mouse or mouse where CFTR is absent (e.g., by transgenic expression of the CFTR mutant gene or by gene targeted mutation of the CFTR gene); (2) isolating genetic material (usually RNA) from the CFTR mutant mouse that encodes the CFTR mutant polypeptide or genetic material from the mouse that does not encode CFTR; and (3) using this isolated genetic material to identify changes in gene expression that compensate for the mutant CFTR or the absence of CFTR.

It is preferred that the mutated CFTR gene be expressed in nonhuman mammals (e.g., mice) that lack endogenous CFTR or are otherwise CFTR-deficient, so that the mutated gene is the only source of CFTR in the tissue of interest, such as lung. The mRNA expression levels in sample(s) from the transgenic mice with mutated CFTR can be analyzed using Affymetrix or other gene chips and compared to the mRNA expression levels in such a sample(s) from wild type mice (i.e., the control) to determine which genes have altered expression levels that can potentially compensate for the impaired CFTR function and/or activity. These potential CFTR modifier genes can then be analyzed further to verify that they alter the homeostasis of the lungs by compensating for loss (or impairment) of CFTR function and/or activity. This verification usually includes expressing the gene in a cell type where the expression of that gene is low or not detectable, and determining the function and/or activity of the polypeptide it encodes. Verification can also include the generation of a transgenic mouse with deletion, misexpression, or overexpression of the gene in question to verify its putative role as a CFTR modifier gene.

Qualitative and quantitative changes in gene expression can also be determined using any of the proteomic assay systems known to those skilled in the art. See, for example, U.S. Pat. No. 6,365,418 (Wagner et al), issued Apr. 2, 2002 (herein incorporated by reference) for arrays of polypeptide (e.g., protein)-capture agents that can be used in such assay systems. Tissues or organs from human or nonhuman mammals described herein can be harvested, fractionated to isolate protein mixtures, and used to assay for qualitative and/or quantitative changes in polypeptide functions and/or activities. Transgenic nonhuman mammals such as CFTR-deficient gut-corrected mice or CFTR-deficient mice expressing mutated CFTR, as well as those with other mutations to the CFTR gene, including gene-targeted mutations, can be used to detect and/or identify CFTR modifier genes using proteomic methodology. An example of a proteomics technology useful for such analyses is the Cyphergen system. Tissues or organs harvested from mice such as the FABP-hCFTR/mCFTR(−/−) mouse can be homogenized and fractionated to isolate a polypeptide mixture; a chip with a polypeptide-binding surface is then contacted with the polypeptide mixture, and the identity of the polypeptides binding to the chip surface is then determined using bioinformatic analysis.

b. Regulators of CFTR Modifier Genes and Polypeptides

The gene regions known in the art as gene promoters can be used in assay systems to detect and/or identify which agents can potentially modulate the expression of a particular CFTR modifier gene. In general, a gene promoter comprises an array of cis-elements recognized and subsequently bound by trans-acting factors produced within a cell. The DNA binding activities of these trans-acting factors modulate(s) expression of all genes within any cell. In general, the trans-acting factors are polypeptides known as transcription factors. Some transcription factors are stimulatory and will increase the level of a gene's expression, while others are repressive, and will reduce the level of expression. Each cell type expresses a cell-specific array of unique or overlapping factors, as compared with other cell types. Each gene promoter requires a specific array of transcription factors. Thus, the phenotype of a cell depends, in great part, upon the array of transcription factors expressed within that cell.

Analysis of promoter elements in a gene promoter can begin with a reporter gene construct, in which a gene promoter is linked to a reporter gene within an expression vector. The reporter gene construct can be introduced into a homogenous population of cultured cells, such as those of an immortalized mammalian cell line, or other suitable cell line or type. If the cells express a sufficient quantity of the required transcription factor(s) required by the promoter in a reporter gene construct, that promoter will cause the reporter gene to be expressed. The amount of polypeptide expressed by the reporter gene can then be measured to determine the level of promoter activity conferred by: (1) the amount or activity of the transcription factor(s); and (2) the presence of the required cis-elements in the promoter sequences.

Since transcription factors are polypeptides, their activity can be modulated or influenced by polypeptide-regulating agents. As a result, a gene promoter can be placed in a reporter gene construct, transfected into a suitable cell type, and used to identify agents that will indirectly modulate the expression of the reporter gene by first affecting the activity of the transcription factors that binds cis-elements of the gene promoter being tested. This provides the ability to develop methods and products for detecting and/or identifying potential genetic regulators that modulate the expression by CFTR modifier genes.

To make a reporter gene, a plasmid cloning vector can be used to build a DNA molecule comprising the gene promoter to be tested, operably-linked to sequences encoding a reporter gene. Also included in the vector are a mammalian intron and SV-40 Poly A sequences. Reporter genes can include chloramphenicol-transferase, LacZ, green fluorescent protein (GFP), luciferase, or any other suitable reporter gene. The construct can be introduced into cells using any one of a variety of techniques well-known to those skilled in the art, including calcium phosphate or calcium chloride co-precipitation, DEAE dextran-mediated transfection, lipofection, or electroporation. Cells can be treated with a single agent, or amplified and plated on 96- or 384-well plates for large-scale screening. This allows the screening of drug libraries or combinatorial libraries to detect and/or identify agents potentially useful as genetic regulators for activating endogenous transcription factors or other cellular processes that modulate gene transcription. Gene promoter sequences can also be sequenced and analyzed using bioinformatics to determine potential cis-elements that are encoded in the promoter sequences.

In one embodiment, promoter regions, such as those of the Kir4.2 gene, are compared with those of CFTR and pulmonary Surfactant Protein-D. Examples of elements common to Kir4.2 and CFTR in the mouse include cAMP Response Element-Binding Protein (CREBP), C-Ets-1, cut-iMe homeodomain protein, hepatic nuclear factor 1, Lentiviral Poly A signal-binding protein, nuclear factor of activated T-cells (NFAT), ocatmer-binding factor 1, Pax-3, PU.1, retroviral Poly A downstream element-binding protein, STAT, and RFX1. As such, these transcription factors are targets of agents that can modulate expression of Kir4.2.

In another embodiment, a reporter gene construct comprising the promoter regions of a CFTR modifier gene such as Kir4.2 can also be transfected into cultured cells to provide an assay to identify genetic regulators of the CFTR modifier gene. Primary cultures of lung epithelial cells or cells from other organs can be harvested from mice described herein and used as assays for identifying agents that can act as genetic regulators for CFTR modifier genes and/or as regulators for CFTR modifier polypeptides. The construct can be introduced into cells using any one of a variety of techniques well-known to those skilled in the art, including calcium phosphate or calcium chloride co-precipitation, DEAE dextran-mediated transfection, lipofection, or electroporation. Cells transfected can include but are not limited to primary, transformed, or immortalized cells. Sources of the cells to be transfected can include but are not limited to mammalian (human and nonhuman), yeast, or insect cells. Cells can be treated with a single agent, or amplified and plated on 96- or 384-well plates for large-scale screening. This allows the screening of drug libraries or combinatorial libraries to detect and/or identify agents potentially useful for activating endogenous transcription factors or other cellular processes that promote CFTR modifier gene transcription.

The polypeptide-encoding regions of CFTR modifier genes, or the polypeptides per se, can be used in assay systems to detect and/or identify agents that can potentially function as polypeptide regulators. Methods and products for detecting and/or identifying polypeptide regulators that use CFTR modifier genes (e.g., the Kir4.2 gene), as well as their expressed polypeptides, include: (1) cell culture assay detection systems for screening potential agents; and (2) proteomic assays of the expressed CFTR modifier polypeptides, following experimental treatment with potential agents.

In cell culture assay detection systems, a CFTR modifier gene can be introduced into and expressed in any of a wide variety of cell types. Cells expressing the CFTR modifier gene can then be used to screen combinatorial libraries or individual compounds or drugs for ones that can influence function and/or activity of the expressed polypeptide (i.e., are polypeptide regulators). Cells transfected can include but are not limited to primary, transformed, or immortalized cells. Sources of the cells to be transfected can include but are not limited to mammalian (human and nonhuman mammal), yeast, or insect cells. The transfection method can be any one of a variety of techniques well-know to those skilled in the art, including calcium phosphate or calcium chloride co-precipitation, DEAE dextran-mediated transfection, lipofection, or electroporation. The transfected cells can be treated with a single agent, or amplified and plated in 96- or 384-well plates for screening on a large scale. CFTR modifier polypeptide activity can be measured or otherwise determined by use of indicator dyes, fluorescence, chemoluminesence, or other indicator methods in response to alterations in ion concentration. Use of indicator dyes, fluorescence, chemoluminesence, or other indicator methods for such purposes are well known to those skilled in the art.

In one embodiment of such a detection and/or identification method, a CFTR modifier gene cDNA nucleotide sequence, such as a mouse Kir4.2 cDNA nucleotide sequence, is placed in an expression vector containing a human cytomegalovirus promoter, a mammalian intron, and a SV-40 poly-A sequence. The Kir4.2-containing expression vector is stably transfected into CHO cells. Kir4.2 mRNA can be detected by RT-PCR, and Kir4.2 polypeptide can be detected by Western Blot, thus verifying that the Kir4.2 cDNA is expressed. The cells can be treated with cAMP-stimulating agents, such as forskolin and/or IBMX. Subsequent to the experimental treatment, increases in potassium (K⁺) ion flow are determined by comparison to that in nontransfected cells.

Qualitative and quantitative changes in expression of CFTR modifier polypeptide levels can also be determined using any of the proteomic assay systems known to those skilled in the art. Agents identified in the above described cell culture assay detection systems can be used for further in vitro or in vivo treatments. The cells can be from any of the cell culture assay systems described herein, or from any of the animals herein described. The cells or tissues can be harvested, fractionated to isolate protein mixtures, and assayed for changes in CFTR modifier polypeptide functions and/or activities in response to experimental treatment. Methods for detecting and/or identifying potential polypeptide regulators include using transgenic nonhuman mammals, such as gut-corrected CFTR-deficient mice, FABP-hCFTR/mCFTR(−/−) or the CFTR-deficient mice expressing mutated CFTR, SPC-hΔ508/FABP-hCFTR/mCFTR(−/−) for proteomic studies, as well as those with other mutations to the CFTR gene, including gene-targeted mutations as model systems for experimental treatments derived from the methods of the present invention. Furthermore, wild type mice and other nonhuman mammal species can also be used as test animals to determine the efficacy of agents that influence CFTR modifier polypeptide function and/or activity. Following experimental administrations of such agents, the organs and tissues of these animals can be harvested and assayed for changes in expression of CFTR modifier polypeptides.

3. Use of CFTR Modifier Genes Polypeptides and Regulators

The present invention further relates to the use of CFTR modifier genes, including up-regulated genes listed in Table 1, as well as down-regulated genes listed in Table 2, their respective expressed polypeptides, genetic regulators of CFTR modifier genes, and/or polypeptide regulators of CFTR modifier polypeptides for treating CF, or at least the conditions that cause CF, including regulating, controlling or otherwise modulating the ion transport of CF-affected cells. The CFTR modifier genes, the respective expressed polypeptides, the genetic regulators and polypeptide regulators can either be used individually to treat CF, or can be used in combination to treat CF. For example, combinations of genetic and polypeptide regulators can be used to treat CF.

a. Genetic Regulators of CFTR Modifier Gene Expression

Suitable genetic regulators for use in the present invention for CFTR modifier genes, and in particular the Kir4.2 gene, include transcription factors (e.g., AP1, PU.1); proto-oncogenes which enhance transcription; interferon gamma and analogues thereof; barbiturates and analogues thereof; NF-κB and analogues thereof; nuclear factor of activated cells and calcium channel activating agents; PU.1 such as ets factor agents and GM-CSF; IL-6, IL-1α, IL-1β, INF-γ and analogues thereof; cAMP analogues, activators of adenylate cyclase and cAMP phosphodiesterase inhibitors; retinoids and orphan receptor activators; retinoic acid receptor agonists, retinols, retinoic acid and analogues thereof; steriodogenic factor, glucocortiods and glucocorticoid analogues, mineralcorticoids, estrogens, progestins, and analogues thereof; betamethasone, Decadron; and mixtures thereof.

It has been found that CFTR modifier genes, and in particular the Kir4.2 gene, can affect, directly or indirectly, ion transport into and out of cells, especially CF-affected cells, and in particular, can affect chloride (Cl⁻) ion transport out of CF-affected cells. In particular, it has been found that the Kir4.2 gene which affects basolateral potassium (K⁺) ion transport, can also potentiate (directly or indirectly) apical chloride (Cl⁻) ion transport. This means that increased stimulation or expression of the Kir4.2 gene can augment chloride (Cl⁻) ion transport in normal and especially CFTR-impaired cells, and can thus bypass CFTR-dependent defects in chloride (Cl⁻) transport and cell function that are believed to be the cause of CF.

The CFTR modifier gene regulator(s) can be formulated as a therapeutic composition or packaged drug for treating a subject having CF. The therapeutic compositions include a therapeutically effective amount of at least one of the aforementioned genetic regulators and optionally a pharmaceutically acceptable carrier. The packaged drug includes at least one of the aforementioned genetic regulators, optionally a pharmaceutically acceptable carrier, and instructions for administering the genetic regulator for treating subjects having CF. The set of instructions can be written or printed on sheet of paper, can be on the packaging associated with the packaged drug, can be in the form of electronic media or software (e.g., floppy disk or CD ROM disk) that can be loaded, installed (directly or by downloading from a remote site such as via a LAN, WAN or the Internet), or otherwise can be read by a computer, personal digital assistant (PDA) or other electronic device, or any other suitable method for providing instructions on how to administer the genetic regulator to treat the subject having CF.

Although the aforementioned genetic regulators can be administered alone, they are preferably administered as part of a pharmaceutical formulation. Such formulations can include pharmaceutically acceptable carriers known to those skilled in the art, as well as other therapeutic agents. It will also be appreciated that the genetic regulators of the present invention can be administered in various pharmaceutically acceptable forms, e.g., as pharmaceutically acceptable salts thereof.

Appropriate dosages of the genetic regulators and formulations administered in accordance with the present invention will depend on the type of CFTR mutation or deficiency, and severity of the condition being treated and can also vary from subject to subject. Determining an acceptable or optimal dosage will generally involve the balancing of the level of therapeutic benefit against any risk or deleterious side effects of the dose and treatment of the present invention. For a dose to be “therapeutically effective,” it must have the desired effect, i.e., modulate the expression of the CFTR modifier polypeptide as defined herein, thus resulting in a beneficial improvement of the subject, for example, improved ion transport (e.g., Cl⁻ secretion) by the CF-affected cell being treated with the dosage. An optimal dose will be one which, when administered to the CF-affected subject, results in, for example, improved ion transport (e.g., chloride (Cl⁻) secretion) at or near wild type CFTR levels.

In addition to the genetic regulator(s), pharmaceutical formulations of the present invention can also comprise additional compounds and/or compositions that will also aid in relief of the symptoms of CF, including a CFTR modifier polypeptide regulator(s) as described hereafter. Pharmaceutical formulations for treating CF are provided that comprise a combination of a safe and therapeutically effective amount of a suitable genetic regulator, as described above, a pharmaceutically acceptable carrier, and a safe and therapeutically effective amount of a CFTR modifier polypeptide regulator(s) as described hereafter. More than one polypeptide regulator can be combined with the genetic regulator(s). The ratio of genetic regulator(s) to polypeptide regulator will depend upon the dose desired of each of the individual compounds. Preferably, the polypeptide regulator will be administered as a pharmaceutically-acceptable aqueous solution wherein the pharmaceutical formulation comprises: (1) from about 0.001% to about 10% of a genetic regulator(s); (2) from about 10% to about 99% of a pharmaceutically-acceptable carrier; and (3) from about 0.001% to about 10% of a polypeptide regulator(s), as described hereafter.

Administration of the genetic regulator(s), with or without a pharmaceutically acceptable carrier(s) and/or additional polypeptide regulator(s), can be by any suitable route including oral, nasal, topical (including buccal and sublingual), parenteral (including subcutaneous, intramuscular, intravenous and intradermal), vaginal or rectal, with oral and nasal administration being preferred. The formulations thus include those suitable for administration through such routes. It will be appreciated that the preferred route can vary with, for example, the condition and age of the subject. The formulations can be conveniently presented in unit dosage form, e.g., tablets and sustained release capsules, and can be prepared and administered by any methods well known to those skilled in the art of pharmacy, including liposomal delivery systems.

Formulations of the present invention suitable for oral administration can be as discrete units such as capsules, cachets or tablets, as a powder or granules, or as a solution, suspension or emulsion. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible carriers. Formulations suitable for oral topical administration further include lozenges, pastilles, mouthwashes and inhalation mists administered in a suitable base or liquid carrier. Lozenge forms can comprise the active ingredient in a flavor, such as sucrose and acacia or tragacanth, as well as pastilles comprising the active ingredient in an inert base, such as gelatin and glycerin or sucrose and acacia emulsions, gels and the like containing, in addition to the active ingredient, such carriers as are known in the art. Formulations suitable for topical administration to the skin can be provided as ointments, creams, gels and pastes comprising the compound to be administered and a pharmaceutically acceptable carrier, or in a transdermal patch.

Formulations suitable for nasal administration wherein the carrier is a solid include powders of a particle size, for example about 20 to 500 microns, which can be administered by rapid inhalation through the nasal passage. Suitable formulations wherein the carrier is a liquid can be administered, for example, as a nasal spray or drops. Formulations suitable for administration by inhalation include aerosol formulations placed into pressurized acceptable propellants, such as dichlorodifluoromethane, trichlorofluoromethane, propane, nitrogen, and the like. The active agent can be aerosolized with suitable excipients. For inhalation administration, the formulation can be dissolved or dispersed in liquid form, such as in water or saline, preferably at a concentration at which the composition is fully solubilized and at which a suitable dose can be administered within an inhalable volume. A suitable dose would place approximately 0.001 to about 5.0 mmol per liter of the composition on the airway surfaces approximately 4 times per day. Delivery can be repeated several times a day, depending upon the specific dosage chosen and the rate at which the chosen composition is cleared from the airways, with the goal being to maintain chloride permeability in the airway epithelial cells. Delivery can be through a nebulizer or a metered-dose inhaler. Suitable methods for aerosol delivery of genetic regulators are also disclosed in U.S. Pat. No. 5,543,399 (Riordan et al), issued Aug. 6, 1996; U.S. Pat. No. 5,641,662 (Debs et al), issued Jun. 24, 1997; U.S. Pat. No. 5,827,703 (Debs et al), issued Oct. 27, 1998; U.S. Pat. No. 5,756,353 (Debs), issued May 26, 1998; U.S. Pat. No. 5,858,784 (Debs et al), issued Jan. 12, 1999; U.S. Pat. No. 5,948,681 (Scanlin et al), issued Sep. 7, 1999; and U.S. Pat. No. 6,001,644 (Debs et al), issued Dec. 14, 1999, all of which are incorporated by reference.

Formulations suitable for parenteral administration include aqueous and non-aqueous sterile injection solutions which can contain anti-oxidants, buffers, bacteriostats and solutes which render the formulation isotonic with the blood of the intended recipient; and aqueous and non-aqueous sterile suspensions which can include suspending agents and thickening agents. Formulations suitable for intravenous and intraperitoneal administration, for example, include aqueous and nonaqueous, isotonic sterile injection solutions, which can contain anti-oxidants, buffers, bacteriostats, and solutes that render the formulation isotonic with the blood of the intended recipient and aqueous and nonaqueous sterile suspensions that can include suspending agents, solubilizers, thickening agents, stabilizers, and preservatives. The formulations can be in unit or multi-dose containers, for example, sealed ampules and vials, and may be lyophilized, requiring only the addition of the sterile liquid carrier such as water for injections immediately prior to use. Extemporaneous injection solutions and suspensions can be prepared from sterile powders, granules and tablets of the kind previously described.

Formulations suitable for vaginal administration can be presented as pessaries, tampons, creams, gels, pastes, foams or spray. Formulations for rectal administration may be presented as a suppository with a suitable base.

b. Regulators of CFTR Modifier Polypeptide Function or Activity

Suitable regulators of function and/or activity of CFTR modifier polypeptides, and in particular Kir4.2 polypeptides, include agents that activate adenylate cyclase in target cells, e.g., adrenergic agents, catacholamines, cAMP agonists and cAMP supplements such as forskolin, isoproterenol and albuterol, cAMP and analogues thereof; various polypeptide hormones, e.g., vasopressin, that stimulate cAMP; cAMP phosphodiesterase inhibitors that block cAMP breakdown such as alkylxanthines, theophylline and aminophylline; cAMP-specific inhibitors such as Rolipram (Shearing AG), glucocorticoid, TGF-β (SMAD₃); potassium K_{ATP} channel openers such as cromakalim, pinacidil, nicorandil, minoxidil sulphate, aprikalim, diazoxide; potassium BK_{Ca} channel openers such as NS004, benzimidazolones such as 1-ethyl-2-benzimidazolinone (1-EBIO), fenamates, dehydroxoyasaponin-I (DHS-I), maxikdiol, cromakalim, nirendipine, and phloretin; UTP, 8-methoxypsoralen (Methoxsalen, 8-MOP), and genistein; calcium ion agonists such as ionomycin, A23187, carbachol, bradykinin, duramycin and thapsigargin; human DNase 1; sodium channel blockers such as amiloride and triamterene; pancreatic enzyme supplements; and mixtures thereof, in dosages useful for relief of the symptoms of CF, as known to those skilled in the art.

Suitable alkylxanthines for use in the present invention include the methylxanthines, such as 3-isobutyl-1-methylxanthine (IBMX) and 1,3-dimethylxanthine (theophylline) and other xanthines such as papaverine, pentoxifilline and caffeine. See also U.S. Pat. No. 5,366,977 (Pollard et al), issued Nov. 22, 1994 (herein incorporated by reference), which discloses compounds that antagonize the A₁-adenosine cell receptor and do not antagonize the A₂-adenosine cell receptor that are suitable for use herein and include 8-cyclopentyl-1,3-dipropylxanthine (CPX), xanthine amino congener (8-[4-[2-aminoethylaminocarbonylmethyloxy]-phenyl]-1,3-dipropylxanthine, XAC), or a therapeutically effective derivative thereof.

Suitable benzimidazole or benzimadazole derivatives for use in the present invention include those disclosed in U.S. Pat. No. 6,159,968 (Cuppoletti), issued Dec. 12, 2000 (herein incorporated by reference), in particular 2-[(pyridyl)-methylsulfinyl or -methylthio]benzimidazole derivatives and salts thereof, for example omeprazole, lansoprazole, thimoprazole and pantoprazole, as well as the following illustrative compounds: 4-trifluoromethyl-2-[(4-methoxy-2-pyridylmethyl)thiol]-(1H)-benzimidazole; 4-trifluoromethyl-2-[(4-methoxy-3-methyl-2-pyridylmethyl)thio]-(1H)-benzimidazole; 4-trifluoromethyl-2-[(4-methoxy-5-methyl-2-pyridylmethyl)thio]-(1H)-benzimidazole; 4-trifluoromethyl-2-[(4-methoxy-3,5-dimethyl-2-pyridylmethyl)thio]-(1H)-benzimidazole; 5-trifluoromethyl-2-[(4-methoxy-2-pyridylmethyl)thio]-(1H)-benzimidazole; 5-trifluoromethyl-2-[(4-methoxy-3-methyl-2-pyridylmethyl)thio]-(1H)-benzimidazole; 5-trifluoromethyl-2-[(4-methoxy-5-methyl-2-pyridylmethyl)thio]-(1H)-benzimidazole; 5-trifluoromethyl-2-[(4-methoxy-3,5-dimethyl-2-pyridylmethyl)thio]-(1H)-benzimidazole; 4-trifluoromethyl-2-[(4-methoxy-2-pyridylmethyl)-sulfinyl]-(1H)-benzimidazole; 4-trifluoromethyl-2-[(4-methoxy-3-methyl-2-pyridylmethyl)sulfinyl]-(1H)-benzimidazole; 4-trifluoromethyl-2-[(4-methoxy-5-methyl-2-pyridylmethyl)sulfinyl]-(1H)-benzimidazole; 4-trifluoromethyl-2-[(4-methoxy-3,5-dimethyl-2-pyridylmethyl)sulfinyl]-(1H)benzimidazole; 5-trifluoromethyl-2-[(4-methoxy-2-pyridylmethyl)sulfinyl]-(1H)-benzimidazole; 5-trifluoromethyl-2-[(4-methoxy-3-methyl-2-pyridylmethyl)sulfinyl]-(1H)-benzimidazole; 5-trifluoromethyl-2-[(4-methoxy-5-methyl-2-pyridylmethyl)sulfinyl]-(1H)-benzimidazole and 5-trifluoromethyl-2-[(4-methoxy-3,5-dimethyl-2-pyridylmethyl)sulfinyl]-(1H)-benzimidazole; 2-[2-(4-methoxy)-pyridylmethylsulfinyl]-(5-acetyl-6-methyl)-benzimidazole; 2-[2-(4-methoxy)-pyridylmethylsulfinyl]-(4,6-dimethyl)-benzimidazole; 2-[2-(3,5-dimethyl-4-methoxy)-pyridylmethylsulfinyl]-(5-acetyl-6-methyl)-benzimidazole; 2-[2-(4-methoxy)-pyridylmethylsulfinyl]-(5-carbomethoxy-6-methyl)-benzimidazole; 2-[2-(4-ethoxy)-pyridylmethylsulfinyl]-(5-carbomethoxy-6-methyl)-benzimidazole; 2-[2-(3-methyl-4-methoxy)-pyridylmethylsulfinyl]-(5-carbomethoxy-6-methyl)-benzimidazole; 2-[2-(3,5-dimethyl-4-methoxy)-pyridylmethylsulfinyl]-(5-carbomethoxy-6-methyl)-benzimidazole; 2-[2-(4-methoxy-5-methyl)-pyridylmethylsulfinyl]-(5-carbomethoxy)-benzimidazole; 2-[2-(3,5-dimethyl-4-methoxy)-pyridylmethylsulfinyl]-5-carbomethoxy)-benzimidazole; 2-[2-(3,5-dimethyl-4-methoxy)-pyridylmethylsulfinyl]-(5-acetyl)-benzimidazole; 2-[2-(4-methoxy-5-methoxy)-pyridylmethylsulfinyl]-(5-methoxy)-benzimidazole; 2-[2-(3,5-dimethyl-4-methoxy)-pyridylmethylsulfinyl]-(5-methoxy)-benzimidazole; 2-[2-(3,5-dimethyl-4-methoxy)-pyridylmethylsulfinyl]-(5-methyl)-benzimidazole; 2-[2-(3,5-dimethyl-4-methoxy)-pyrdylmethysulfinyl]-benzimidazole; 2-[2-(3,5-dimethyl-4-methoxy)-pyridylmethylsulfinyl]-(5-chloro)-benzimidazole; 2-[2-[3-methyl-4-(2,2,2-trifluoroethoxy)pyridyl]methylsulfinyl]benzimidazole(lansoprazole); 2-[2-[3-methyl-4-(2,2,3,3-tetrafluoropropoxy)pyridyl]methylthio]benzimidazole; 2-[(2-pyridyl)methylsulfinyl]benzimidazole(thimoprazole); 2-[2-(3,5-dimethyl-4-methoxypyridyl)methylsulfinyl]-5-methoxy-1H-benzimidazole (omeprazole); 2-[2-[4-(3-methoxypropoxy)-3-methylpyridyl]methylsulfinyl]-1H-benzimidazole; 2-[2-(3,4-dimethoxypyridyl)methylsulfinyl]-5-difluoromethoxy-1H-benzimidazole (pantoprazole); 4-methyl-3-(2,2,2-trifluoroethoxy)-5H-pyrido[1′,2′:4,5 [1,2,4]thiaziano[2,3-a]benzimidaxole-13-ium tetrafluoroborate or a pharmaceutically acceptable salt thereof.

Like the CFTR genetic regulators, the CFTR modifier polypeptide regulator(s) can be formulated as a therapeutic composition or packaged drug for treating a subject having CF. The therapeutic compositions include a therapeutically effective amount of at least one of the aforementioned polypeptide regulators and a pharmaceutically acceptable carrier. The packaged drug includes at least one of the aforementioned polypeptide regulator(s) and instructions for administering the polypeptide regulator for treating subjects having CF. The set of instructions can be written or printed on sheet of paper, can be on the packaging associated with the packaged drug, can be in the form of electronic media or software (e.g., floppy disk or CD ROM disk) that can be loaded, installed (directly or by downloading from a remote site such as via a LAN, WAN or the Internet), or otherwise be read by a computer, personal digital assistant (PDA) or other electronic device, or any other suitable method for providing instructions on how to administer the genetic regulator to treat the subject having CF.

Appropriate dosages of the CFTR modifier polypeptide regulators and formulations administered in accordance with the present invention will depend on the type of CFTR mutation or deficiency, and severity of the condition being treated and can also vary from subject to subject. Determining an acceptable or optimal dosage will generally involve the balancing of the level of therapeutic benefit against any risk or deleterious side effects of the dose and treatment of the present invention. For a dose to be “therapeutically effective,” it must have the desired effect, i.e., influence the function and/or activity of the CFTR modifier polypeptide as defined herein, thus resulting in a beneficial improvement of the subject, for example, improved ion transport (e.g., chloride (Cl⁻) secretion) by the CF-affected cell being treated with the dosage. An optimal dose will be one which, when administered to the CF-affected subject, results in, for example, improved ion transport (e.g., chloride (Cl⁻) secretion) at or near wild type CFTR levels.

Suitable methods of administration, pharmaceutical formulations, pharmaceutically acceptable carriers, etc., for these CFTR modifier polypeptide regulators can be the same or similar as those used for the genetic regulators, as previously described. Suitable methods for aerosol delivery of CFTR modifier polypeptide regulators are also disclosed in U.S. Pat. No. 5,543,399 (Riordan et al), issued Aug. 6, 1996; U.S. Pat. No. 5,641,662 (Debs et al), issued Jun. 24, 1997; U.S. Pat. No. 5,827,703 (Debs et al), issued Oct. 27, 1998; U.S. Pat. No. 5,756,353 (Debs), issued May 26, 1998; U.S. Pat. No. 5,858,784 (Debs et al), issued Jan. 12, 1999; U.S. Pat. No. 5,948,681 (Scanlin et al), issued Sep. 7, 1999; and U.S. Pat. No. 6,001,644 (Debs et al), issued Dec. 14, 1999, all of which are incorporated by reference.

c. CFTR Modifier Polypeptide Therapy

Polypeptide therapy can be accomplished by any method that effectively introduces CFTR modifier polypeptides into the membrane of CF-affected cells to impart to those cells CFTR modifier function and/or activity. An effective amount of a CFTR modifier polypeptide (i.e., an amount sufficient to reduce, alleviate, ameliorate, or otherwise improve the symptoms associated with CF) can be administered alone or in association with an agent that facilitates passage (e.g., via fusion or endocytosis) through cell membranes to CF subjects (i.e., subjects having CF defective cells). What is an “effective amount” can be determined by one skilled in the art based on such factors as the type and severity of symptoms being treated, the weight and/or age of the subject, the previous medical history of the subject, and the selected route for administration of the agent.

Recombinant or native CFTR modifier polypeptide can be purified from host cells using known methods, such as ion exchange chromatography, gel filtration chromatography, electrophoresis and affinity chromatography. See Tilly et al, J. Biol. Chem., (1992) 267(14):9470-73; Anderson et al, Science (1991) 251:679-682). One embodiment of method of purification involves first solubilizing the protein in the presence of a nondenaturing detergent.

For use in protein therapy, CFTR modifier polypeptides typically are associated with lipids, such as detergents or other amphipathic molecule micelies, membrane vesicles, liposomes, virosomes, or microsomes. Lipid compositions that are naturally fusogenic or can be engineered to become fusogenic (e.g. by incorporating a fusion protein into the lipid) are especially preferred. Fusion proteins can be obtained from viruses such as parainfluenza viruses 1-3, respiratory syncytial virus (RSV), influenza A, Sendai virus, and togavirus fusion protein. Nonviral fusion proteins include normal cellular proteins that mediate cell-cell fusion. Other nonviral fusion proteins include the sperm protein PH-30 which is an integral membrane protein located on the surface of sperm cells that is believed to mediate fusion between the sperm and the egg. See Blobel et al, Nature (1992) 356:248-251. Still other nonviral fusion proteins include chimaeric PH-30 proteins such as PH-30 and the binding component of hemaglutinin from influenza virus and PH-30 and a disintegrin (e.g. bitistatin, barbourin, kistrin, and echistatin). In addition, lipid membranes can be fused using traditional chemical fusogens such as polyethylene glycol (PEG).

Like the CFTR genetic regulators, the CFTR modifier polypeptide(s) can be formulated as a therapeutic composition or a packaged drug for treating a subject having CF. The therapeutic compositions include a therapeutically effective amount of at least one of the aforementioned CFTR modifier polypeptides and a pharmaceutically acceptable carrier. The packaged drug includes at least one of the aforementioned CFTR modifier polypeptides and instructions for administering the polypeptide regulator for treating subjects having CF. The set of instructions can be written or printed on sheet of paper, can be on the packaging associated with the packaged drug, can be in the form of electronic media or software (e.g., floppy disk or CD ROM disk) that can be loaded, installed (directly or by downloading from a remote site such as via a LAN, WAN or the Internet), or otherwise be read by a computer, personal digital assistant (PDA) or other electronic device, or any other suitable method for providing instructions on how to administer the genetic regulator to treat the subject having CF.

Appropriate dosages of the CFTR modifier polypeptides and formulations administered in accordance with the present invention will depend on the type of CFTR mutation or deficiency, and severity of the condition being treated and can also vary from subject to subject. Determining an acceptable or optimal dosage will generally involve the balancing of the level of therapeutic benefit against any risk or deleterious side effects of the dose and treatment of the present invention. For a dose to be “therapeutically effective,” it must have the desired effect, i.e., providing a beneficial improvement for the subject, for example, improved ion transport (e.g., Cl⁻ secretion) by the CF-affected cell being treated with the dosage. An optimal dose will be one which, when administered to the CF-affected subject, results in, for example, improved ion transport (e.g., chloride (Cl⁻) secretion) at or near wild type CFTR levels.

Suitable methods of administration, pharmaceutical formulations, pharmaceutically acceptable carriers, etc., for these CFTR modifier polypeptides can be the same or similar as those used for the genetic and polypeptide regulators, as previously described. Suitable methods for aerosol delivery of CFTR modifier polypeptides are also disclosed in U.S. Pat. No. 5,543,399 (Riordan et al), issued Aug. 6, 1996; U.S. Pat. No. 5,641,662 (Debs et al), issued Jun. 24, 1997; U.S. Pat. No. 5,827,703 (Debs et al), issued Oct. 27, 1998; U.S. Pat. No. 5,756,353 (Debs), issued May 26, 1998; U.S. Pat. No. 5,858,784 (Debs et al), issued Jan. 12, 1999; U.S. Pat. No. 5,948,681 (Scanlin et al), issued Sep. 7, 1999; and U.S. Pat. No. 6,001,644 (Debs et al), issued Dec. 14, 1999, all of which are incorporated by reference.

d. CFTR Modifier Genes Therapy

The present invention further relates to the delivery of CFTR modifier genes, with or without other agents or treatments, as a therapy for treating CF. See, for example, U.S. Pat. No. 5,240,846 (Collins et al), issued Aug. 31, 1993, and U.S. Pat. No. 5,958,893 (Welsh et al), issued Sep. 28, 1999 (herein incorporated by reference), which describe suitable methods for delivering CFTR modifier genes according to the present invention. Recombinant retroviral vectors as well as other CFTR gene transfer schemes can be used in the practice of the present invention. Both CF epithelial cells and cell lines that carry a CFTR modifier gene transducted or transferred therein can be used.

CFTR modifier genes used in therapies for treating CF can be obtained through conventional methods such as DNA cloning, artificial construction or other means. Gene transfer for such therapies can be accomplished through a variety of means well known in the art, including transfection using calcium phosphate co-precipitation, fusion of the target cell with liposomes, erythrocyte ghosts or spheroplasts carrying the CFTR modifier gene, plasmid and viral vector-mediated transfer and DNA protein complex-mediated gene transfer.

Alternatively, a preparation of the gene encoding a CFTR modifier polypeptide can be incorporated into a suitable vector for delivering the gene into a CF subject's CF-affected cells. CFTR modifier genes encoding the appropriate polypeptide (for example, in a suitable expression cassette as described hereafter), can be introduced into cells in culture using standard techniques (e.g., via calcium phosphate or calcium chloride co-precipitation, DEAE dextran mediated transfection, lipofection, or electroporation). Recombinant cells can then be cultured in vitro in a manner that allows expression of the CFTR modifier polypeptide. Preferred host cells for generating CFTR modifier polypeptides include for example, mammalian (human and nonhuman mammals) cells, yeast cells and insect cells.

A recombinant viral vector useful in the such therapies comprises DNA of at least a portion of the retroviral genome which portion is capable of infecting the target cells and a CFTR modifier gene operatively linked thereto. By “infection” is generally meant the process by which a virus transfers genetic material to its host or target cell. Preferably the retrovirus used in the construction of a vector of the present invention is also rendered replication-defective to remove the effects of viral replication on the target cells. In such cases, the replication-defective viral genome can be packaged by a helper virus in accordance with conventional techniques. Generally any retrovirus meeting the above criteria of infectiousness and capabilities of CFTR gene transfer can be employed in the practice of the present invention. It will be appreciated that when viral vector schemes are employed for CFTR modifier gene transfer, the use of attenuated or a virulent virus can also be desirable. Where applicable in the practice of the invention, amplification of the CFTR modifier gene can also be utilized to enhance the levels of normal expression.

The cells targeted for transduction or gene transfer in accordance with the present invention include any cells to which the delivery of the CFTR modifier gene is desired. Generally speaking, the cells are those with the CFTR gene defect or deficiency, such as CF-affected cells. The CF-affected cells targeted are preferably epithelial cells, including pancreatic, sweat gland, liver, intestinal, kidney and even more preferably epithelial airway cells, such as lung cells.

Cells or cell populations can be treated in accordance with the present invention in vivo or in vitro. For example, in in vivo treatments, CFTR modifier vectors of the present invention can be administered to the subject, preferably in a biologically compatible solution or pharmaceutically acceptable delivery vehicle, by ingestion, injection, inhalation or any number of other methods. The dosages administered will vary from subject to subject and will be determined by the level of enhancement of CFTR function balanced against any risk or deleterious side effects. Monitoring levels of transduction, CFTR modifier expression and/or the presence or levels of CFTR modifier polypeptide will assist in selecting and adjusting the dosages administered. In vitro transduction is also contemplated by the present invention. Cell populations with defective CFTR genes can be removed from the subject or otherwise provided, transduced with a CFTR modifier gene in accordance with the principles of the present invention, then (re)introduced into the subject.

Although any CF-affected epithelial cells such as pancreatic and sweat gland cells can be targeted with the gene transfer methods and vectors of the present invention, because the most severe complications of CF are usually pulmonary, airway epithelial cells are the most desirable targets for gene therapy of the present invention. Moreover, given that airway epithelial cells have been found to be easily infected by recombinant retroviruses, gene transfer in accordance with the present invention to these cells is quite feasible.

Suitable methods for aerosol delivery of CFTR modifier genes, as well as methods for transfecting cells, are also disclosed in U.S. Pat. No. 5,543,399 (Riordan et al), issued Aug. 6, 1996; U.S. Pat. No. 5,641,662 (Debs et al), issued Jun. 24, 1997; U.S. Pat. No. 5,827,703 (Debs et al), issued Oct. 27, 1998; U.S. Pat. No. 5,756,353 (Debs), issued May 26, 1998; U.S. Pat. No. 5,858,784 (Debs et al), issued Jan. 12, 1999; U.S. Pat. No. 5,948,681 (Scanlin et al), issued Sep. 7, 1999; and U.S. Pat. No. 6,001,644 (Debs et al), issued Dec. 14, 1999, all of which are incorporated by reference.

An “expression cassette” comprising the CFTR modifier gene encoding the appropriate polypeptide operably linked or under the control of transcriptional and translational regulatory elements (e.g. a promoter, ribosome binding site, operator, or enhancer) can be made and used for expression of the CFTR modifier polypeptide in vitro or in vivo. The choice of regulatory elements employed can vary, depending, for example, on the host cell to be transfected and the desired level of expression. Several gene promoters for use in mammalian cells are known in the art and include, for example, the surfactant protein-C (SP-C) promoter for lung specific expression, the phosphoglycerate (PGK) promoter, the simian virus 40 (SV 40) early promoter, the Rous sarcoma virus (RSV) promoter, the adenovirus major late promoter (MLP) and the human cytomegalovirus (CMV) immediate early I promoter. However, any gene promoter that facilitates suitable expression levels can be used in the present invention. Inducible gene promoters, (e.g. those obtained from the heat shock gene, metallothionein gene, beta interferon gene, or steroid hormone responsive genes) can be useful for regulating transcription based on external stimuli.

4. Identification of CFTR Modifier Genes Including the Kir4.2 Gene

The present invention is also directed at the detection and identification of CFTR modifier genes, including identifying RNAs influenced by the presence or absence of CFTR in vivo, to identify genes, including up-regulated genes like those listed in Table 1 and down-regulated genes like those listed in Table 2, and to identify pathways that interact with or compensate for CFTR to maintain or normalize pulmonary function. Stereotypic genomic responses to the lack of CFTR are observed in pulmonary tissues in the absence of infection or disease.

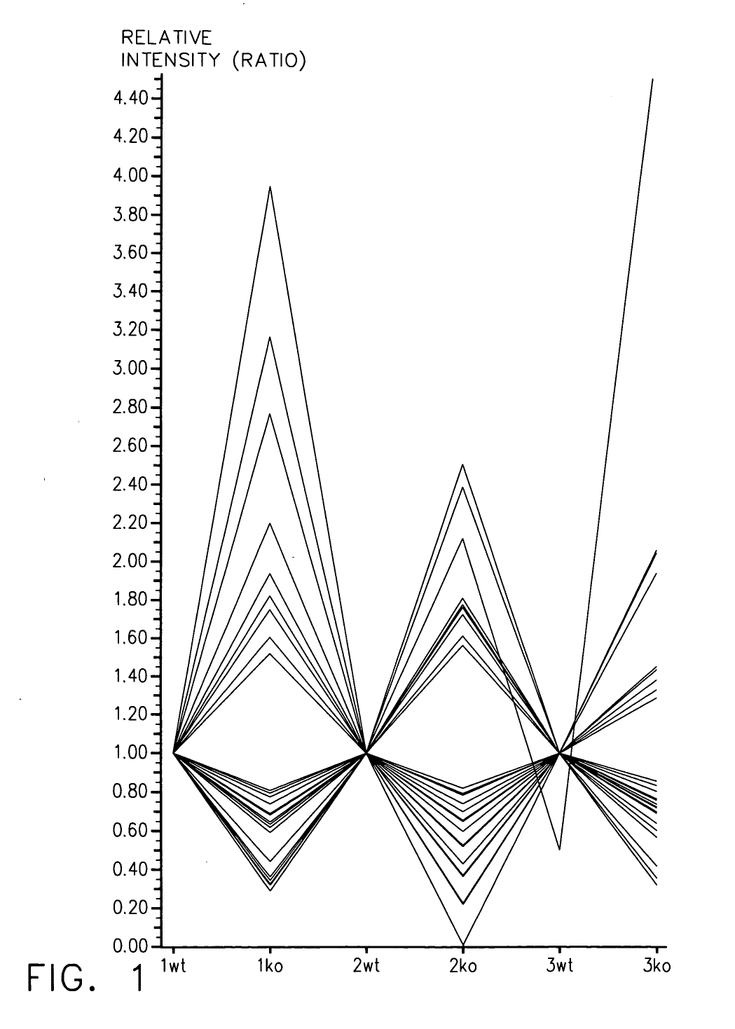

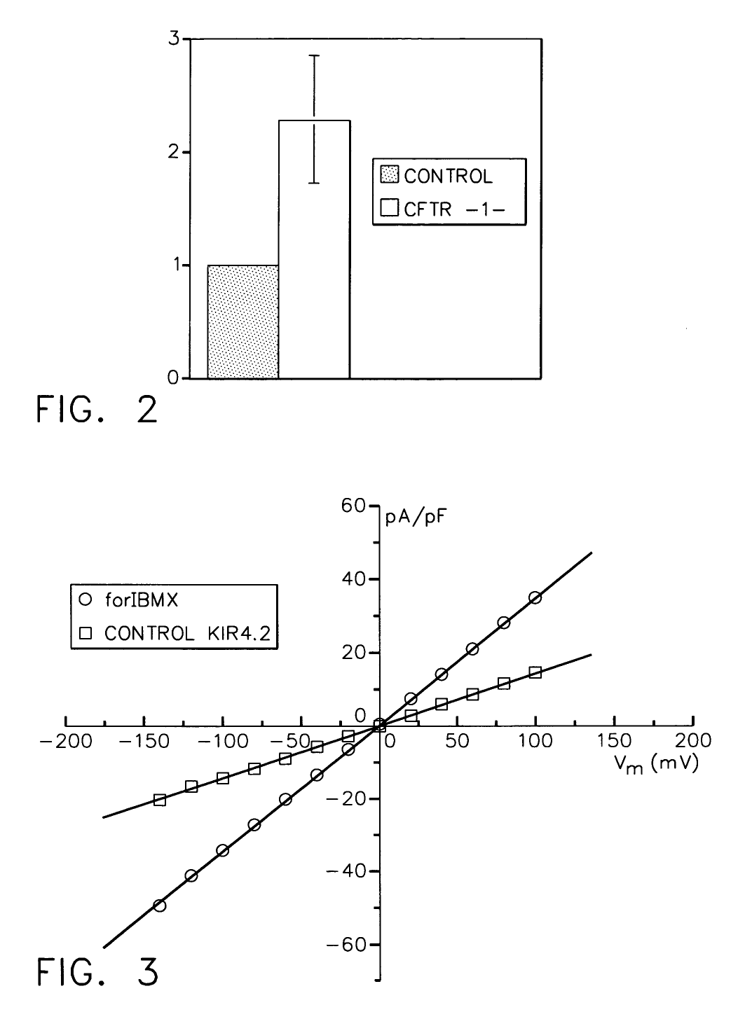

In one embodiment, Affymetrix mouse gene arrays are used to detect differential expression (relative intensity plotted on y-axis v. pairs of mice of increasing age on x-axis) of lung mRNAs isolated from age-matched wild-type and CFTR-deficient mice, specifically CFTR(+/+) versus FABP-hCFTR/mCFTR(−/−) or CFTR(−/−) mice. A CFTR-deficient mouse expressing mutated CFTR, SPC-hΔ508/FABP-hCFTR/mCFTR(−/−), is also analyzed in the same manner, as well as mice with other mutations to the CFTR gene, including doxycyline-induced mutations. Bioinformatic filtering analysis (i.e., using p-value<0.02) revealed 341 genes that are statistically correlated with the CFTR-deficient phenotypes. Expression of CFTR itself is markedly decreased in the CFTR(−/−) mice, validating the mouse model. With additional filtering, twenty-seven of these 341 genes met statistical tests (p-value<0.05) that validated their difference in CFTR-deficient mice as compared to wild type, suggesting that these genes can potentially modify CFTR-dependent pathways, and therefore, the CF disease process (see FIG. 1). The basis for the compensatory activity of one of these 27 genes, Kir4.2, is increased in all CFTR-deficient mice tested. Kir4.2 mRNA is also increased in CFTR-deficient mice compared to wild type mouse lungs as assessed by LightCycler PCR, confirming the gene array data (see FIG. 2). See Gosset et al, “A New Inward Rectifier Potassium Channel Gene (KCNJ15) Localized on Chromosome 21 in the Down Syndrome Chromosome Region 1 (DCR 1),” Genomics (1997) 44:237-41. A mouse Kir4.2 cDNA is expressed in CHO cells, which are then treated with cAMP-stimulating agents, such as combinations of forskolin and IBMX, which increased potassium (K⁺) ion flow (see FIG. 3). Since basolateral potassium (K⁺) transport potentiates apical chloride (Cl⁻) transport, this suggests that increased stimulation or expression of Kir4.2 can potentially augment chloride (Cl⁻) ion transport in normal or CFTR-impaired cells or lungs. Since Kir4.2 is expressed in the relevant regions of the pulmonary airway epithelium, increased expression of Kir4.2 can be used to bypass CFTR-dependent defects in chloride (Cl⁻)⁻ transport and cell function.

Another embodiment involving the detection and identification of CFTR modifier genes is as follows: Human CFTR cDNA is expressed in the intestinal epithelium under control of the intestinal fatty acid binding protein gene promoter (iFABP), fully correcting small intestinal pathology and supporting normal postnatal survival of CFTR (−/−) transgenic mice. The iFABP-hCFTR, CFTR (−/−) mice can be maintained in a mixed FVB/N, C57BL/6 background without evidence of GI or pulmonary disease. Histological and biochemical studies identify no overt pathology in lung tissue from these mice compared to CFTR-expressing littermate controls. See Zhou et al, Science, (1994), 266:1705-8; Chroneos, J. Immunol., (2000) 165:3941-50. Mice are housed in microisolator cages. Lungs of adult iFABP-hCFTR, CFTR (−/−) and control mice are free of bacterial pathogens or colonization as assessed by quantitative culture of lung homogenates on blood agar plates.

Matings of FABP-hCFTR (+/+)/mCFTR (−/−) mice to wild type FVB/N-mCFTR (+/+) mice, are used to produce F1 FABP-hCFTR (±)/mCFTR (∓) mice. These mice are crossed to generate F2 offspring littermates which are then genotyped. Genotyping is performed using the following primers: primers for mCFTR PCR are forward primer (intron 9): 5′-AGG GGC TCG CTC TTC TTT GTG AAC, -3′ reverse primer (intron 10): 5′-TGG CTG TCT GCT TCC TGA CTA TGG, -3′ for neomycin resistance gene PCR are forward primer: 5′-CAC AAC AGA CAA TCG GCT GCT, -3′ and reverse primer: 5′-ACA GTT CGG CTG GCG CGA G, -3′ and for hCFTR PCR are forward primer (exon 9): 5′-AAA CTT CTA ATG GTG ATG ACA G-3′. Reverse primer (exon 11): 5′-AGA AAT TCT TGC TCG TTG AC-3′. FABP-hCFTR(+/+)/mCFTR (−/−) and hCFTR (+/+)/mCFTR (+/+) mice are identified. All CFTR (+/+) mice are heterozygous for the targeted mCFTR gene.

In the following examples, cDNA synthesis and microarray analysis are performed in pairs to minimize technical variability related to RNA isolation and hybridization conditions. Lungs from sex-matched littermates are carefully dissected and the conducting airways and mediastinal structures removed. Lungs are homogenized in TRIzol reagent (Life Technologies) using methods recommended by the manufacturer. In order to minimize the potential influence of strain differences in this mouse colony, lung RNA is isolated from sex matched littermates at 3, 6, and 11 weeks of age. RNA is also isolated from lungs of surviving CFTR (−/−)/hCFTR (−/−) and CFTR (+/+) littermates at 3 weeks of age for comparison with those bearing the iFABP-hCFTR transgene.

### EXAMPLE 1

Total RNA is subjected to reverse transcription using oligo dT with T7 promoter sequences, followed by second strand cDNA synthesis. Antisera cRNA is then amplified and biotinylated using T7 RNA polymerase, prior to hybridization to the Affymetrix genechip Mouse U74aV2 using the Affymetrix recommended protocol. Affymetrix MicroArray Suite version 5.0 is used to scan and quantitate the genechips using default scan settings. Intensity data is collected from each chip, scaled to a target intensity of 1500 and the results are analyzed using both MicroArray Suite and GeneSpring 5.0 (Silicon Genetics, Inc., Redwood City, Calif.). cDNAs are hybridized to U74aV2 chips (Affymetrix Inc.). Hybridization data are normalized in a 2-step process to remove or minimize systemic sources of variation at both chip and gene level. Specifically, each chip is normalized to the distribution of all genes on the chip to control for variation between samples. Each RNA sample from mCFTR (−/−) mice is normalized to its specific control (i.e., sex and age-matched mCFTR (+/+) littermates). Data are further transformed into log ratio for analysis and symmetry of distribution. Changes in RNA levels are identified by the combination of a distribution analysis (JMP4, SAS Institute, Inc.), and the Welch ANOVA. Outlier box and quartile box plots are used to identify outliers with the definition of up-outlier>=upper quartile+1.5 (interquartile range), and the down-outlier<=lower quartile−1.5 (interquartile range). Significant changes are calculated by Welch t-test at p value<=0.05. Adjusted P-values are calculated by Westfall and Young permutation for correction of false positives (GeneSpring 4.2.1, Silicon Genetics). Comparisons between each genotype and age groups are performed using one-way ANOVA. To identify genes that are differentially expressed because of CFTR genotype regardless of age, hierarchical and k-means clustering are used to identify consistent changes in gene expression in response to the lack of CFTR at all three time points. Candidate RNAs are further filtered on the basis of reproducibility and absolute intensity. Mean, standard deviation and coefficient variation are calculated for each replicate. Replicates with CV>=50% are deleted from analysis. Genes whose expression is below level of detection are eliminated as experimental noise.

Results:

To identify genes responsive to CFTR, lung RNAs from iFABP-hCFTR, mCFTR (−/−), iFABP-hCFTR, mCFTR (+/+); mCFTR (−/−) and mCFTR (+/+) littermates at the age of 3, 6 or 11 weeks of age are compared. Microarray analyses are performed in duplicate from RNA isolated at 3 and 6 weeks of age. Data from ten Affymetrix Murine Genome U74Av2 chips are normalized and statistical differences between CFTR deficient (CFTR−) and control (CFTR+) mice are identified. Differences related to age are identified by outlier analysis and/or unpaired t-test. After normalization, normal distributions are observed in the intensity data from lung tissue obtained at all ages. Lung RNA data from 3 week old CFTR (+/+) and CFTR (−/−) mice (lacking the iFABP-hCFTR transgene) are similarly distributed to those bearing the FABP-hCFTR gene and are, therefore, included in the analysis.

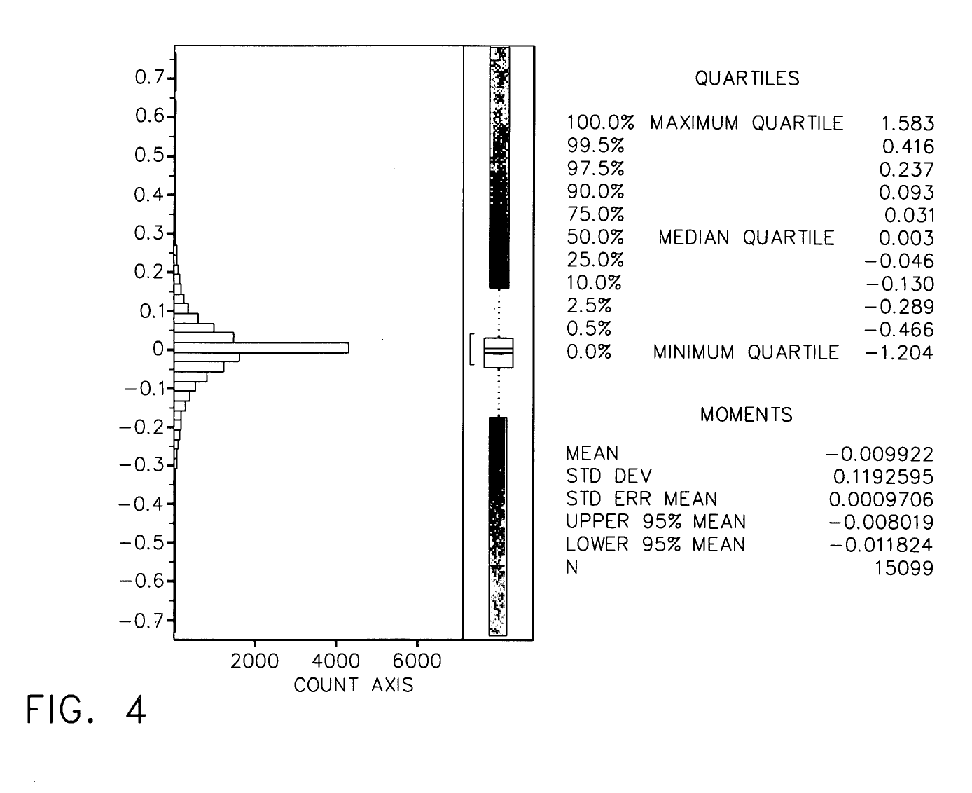

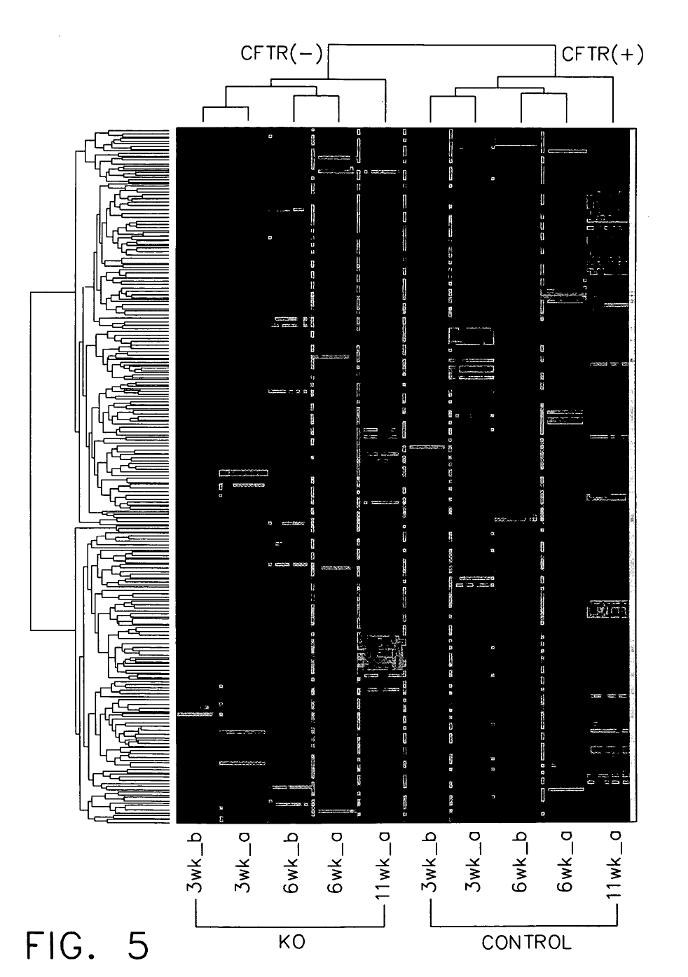

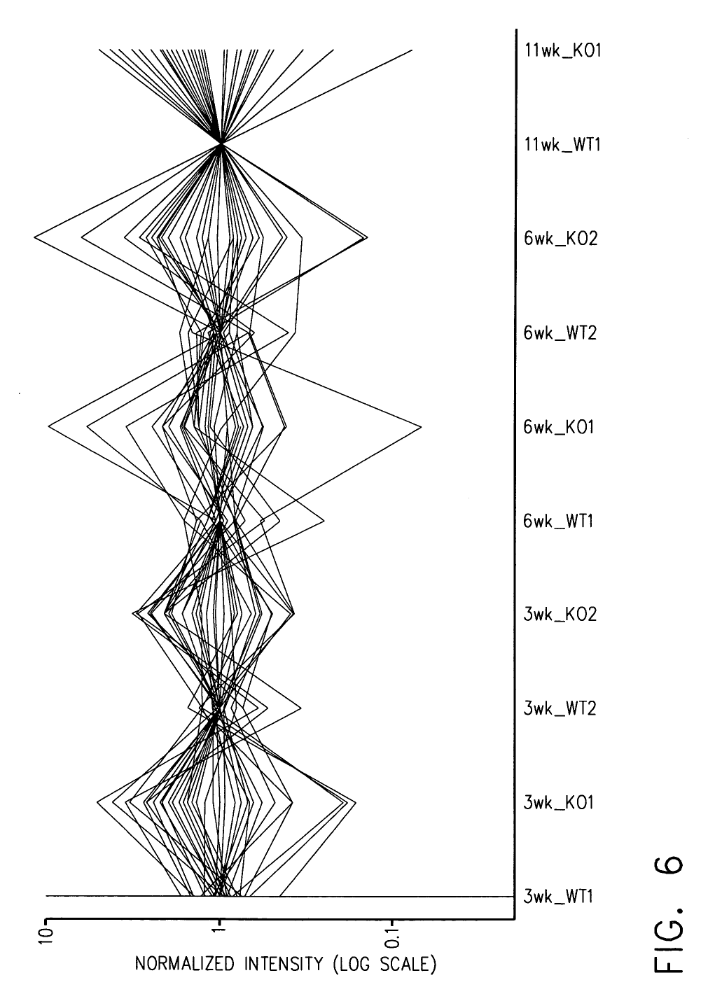

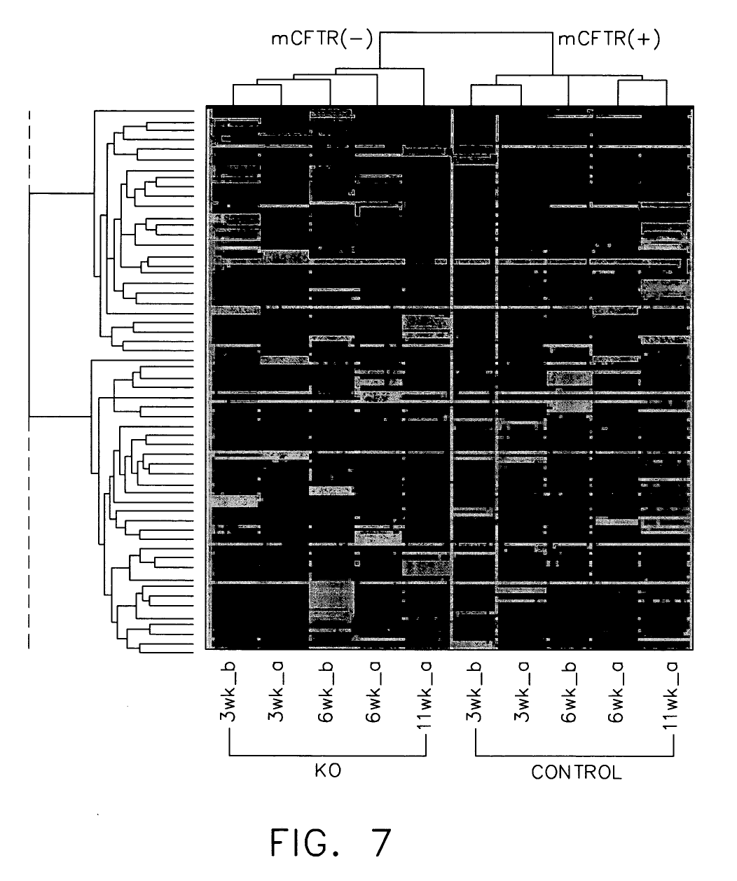

To identify RNAs that are differentially expressed in response to CFTR regardless of age, CFTR (−/−) and CFTR (+/+) data are separated into two groups. The log-ratio distribution and outlier plot of the combined data set are represented by FIG. 4. A total of 1977 outliers are identified from 12442 genes/ESTs analyzed. The abundance of 848 RNAs is increased; 1129 are decreased. Welch t-test together with Westfall and Young step-down permutation further narrow the number of differentially expressed RNAs to 315. Hierarchical clustering is used to visualize and classify the data set. See FIG. 5. Data are shown in a 2D matrix to identify groups of genes with similar expression patterns and show remarkably ordered gene expression profiles of 315 selected genes. On the chip level (top dendrogram) RNAs influenced by CFTR form two distinct groups. Within each group, the samples collected from age matched pairs are more closely related than those from different ages, suggesting that age also influences gene expression. At the RNA level (the dendrogram at the left side), genes are clearly separated into two major groups: those mRNAs increased or decreased in mCFTR (−/−) mice. Genes are further filtered for the consistency of differences in expression levels across all time points (CV<=50%) and for their absolute intensity above 243 (90% of genes called absent by Affymetrix software<=243 for this data set). Additional filters reduce the number of RNAs to 54, of which 29 are consistently increased and 25 which are decreased in mCFTR(−/−) compared to their mCFTR (+/+) littermates. See Table 1 (Up-Regulated Genes) and Table 2 (Down-Regulated Genes). The expression profiles of these 54 genes are shown in FIGS. 6 and 7, demonstrating consistent patterns of expression of the CFTR responsive RNAs regardless of age.

Differentially expressed genes are further classified according to their known or predicted functions. Each gene is annotated and assigned to a functional category. To simplify the calculation, genes in each category are assumed to fit to a binomial distribution. The binomial probability is calculated for each category using entire U74Av2 as reference dataset. “Inflammatory Response” is the most represented category of those RNAs increased in mCFTR (−/−) mice. Among RNAs whose abundance is increased by the lack of CFTR, those influencing inflammation, transcription, and transport are most highly represented and consist of a group of functional categories quite distinct from those whose expression is decreased in mCFTR (−/−) mice. See Table 1 (Up-Regulated Genes) and Table 2 (Down-Regulated Genes). The potential influence of the FABP-hCFTR transgene on RNA expression is also assessed using Welch t-test at the three ages. Differentially regulated RNAs identified in analyses of GI-corrected mice are similarly affected in mCFTR (−/−) mice, demonstrating a lack of effect of iFABP-hCFTR on this subset of genes. Genes whose expression is independently altered by the iFABP-hCFTR transgene include 7 RNAs that decreased and 11 that increased. Differences in their levels of expression are modest (less than 1.5-fold) as shown in the following table:

TABLE 3
Effect of fabp-hCFTR (+/+) on Lung Gene Expression

| Gene | P-value | Ratio | Common | GeneBank | BioProcess |
|---|---|---|---|---|---|
| transmembrane 9 superfamily | 0.0381 | −1.13 | Tm9sf2 | NM_080556 | transport |
| member 2 | | | | | |
| mitochondrial ribosomal protein | 0.03333 | −1.19 | Mrps24 | AA543858 | unknown |
| S24 | | | | | |
| oncostatin receptor | 0.02857 | −1.26 | Osmr | NM_011019 | signal transduction |
| RIKEN cDNA 4432409D24 gene | 0.02857 | −1.17 | 4432409D24Rik | AK014481 | unknown |
| RIKEN cDNA 5730533P17 gene | 0.02857 | −1.32 | 5730533P17Rik | NM_027492 | unknown |
| expressed sequence AI323512 | 0.02857 | −1.22 | AI323512 | AI323512 | unknown |
| nuclear factor I/B | 0.02857 | −1.17 | Nfib | NM_008687 | transcription |
| | | | | | regulation\| |
| cold shock domain protein A | 0.02381 | −1.25 | Csda | NM_139117 | transcription |
| | | | | | regulation\| |
| aldehyde dehydrogenase 9, | 0.02857 | −1.20 | Aldh9a1 | NM_019993 | oxidoreductase |
| subfamily A1 | | | | | |
| heat shock protein, 105 kDa | 0.04762 | −1.40 | Hsp105 | NM_013559 | heat shock response |
| RIKEN cDNA 2610318I15 gene | 0.02381 | −1.14 | 2610318I15Rik | AK012045 | serine/threonine |
| | | | | | kinase |
| caspase 3, apoptosis related | 0.04762 | 1.13 | Casp3 | NM_009810 | apoptosis |
| cysteine protease | | | | | |
| ubiquitin-conjugating enzyme 8 | 0.02857 | 1.68 | Ubce8 | NM_019949 | protein modification |
| T-cell receptor alpha chain | 0.02857 | 1.49 | Tcra | U07662 | cellular defense |
| | | | | | response |
| protein tyrosine kinase 2 beta | 0.02857 | 1.31 | Ptk2b | BF579309 | Kinase |
| thymine DNA glycosylase | 0.02857 | 1.17 | Tdg | NM_011561 | DNA repair |
| caspase 7 | 0.02381 | 1.23 | Casp7 | NM_007611 | apoptosis |
| chemokine (C-C) receptor 7 | 0.02381 | 1.47 | Cmkbr7 | NM_007719 | chemotaxis |

Lung RNA samples from iFABP-hCFTR,CFTR(−/−) (i.e., gut corrected, GC) were compared with those from CFTR(+/+) (lacking the iFARP-hCFTR transgene, i.e., gut uncorrected, NGC).

Ratio is defined as: R=GC/NGC if (GC>NGC); R=−NGC/GC if (GC<NGC).

### EXAMPLE 2

To validate the responsive RNAs identified by microarray analyses, real time RT-PCR is performed using a Light cycler® or a regular thermo cycler followed by gel electrophoresis. Lung RNAs are isolated as described above. cDNAs are generated by reverse transcription and PCR analysis is performed using the following primers for: Kir 4.2 forward 5′-CTT TGA GTT TGT GCC TGT GGT TTC, -3′ reverse 5′-GCT GTG TGA TTT GGT AGT GCG G, -3′; human CFTR same as above; and mouse CFTR forward 5′-TGC TTC CCT ACA GAG TCA TCA ACGG, -3′, reverse 5′-CAC AGG ATT TCC CAC AAC GCA GAG-3′; and B-actin for normalization forward 5′-TGG AAT CCT GCG GCA TCC ATG AAC; reverse 5′-TAA AAC GCA GCT CAG TAA CAG TCC G, -3′; and GAPDH forward 5′-CTT CAC CAC CAT GGA GAA GGC, -3′ reverse 5′-GGC ATG GAC TGT GGT CAT GAG, -3′; and CEBP8 forward 5′-CGC AAC AAC ATC GCT GTG, -3′ reverse 5′-GGG CTG GGC AGT TTT TTG;-3′ and TNF-AIP-3 forward 5′-GCA CGA ATA CAA GAA ATG GCA GG, -3′ reverse 5′-GGC ATA AAG GCT GAG TGT TCA, -3′ CG; and Grin 2d forward 5′-CCT TCT TTG CCG TCA, TCT TTC TTG C, -3′ reverse 5′-AAA CTT CAG GGG TGG GTA TTG CTC C, -3′.

Results:

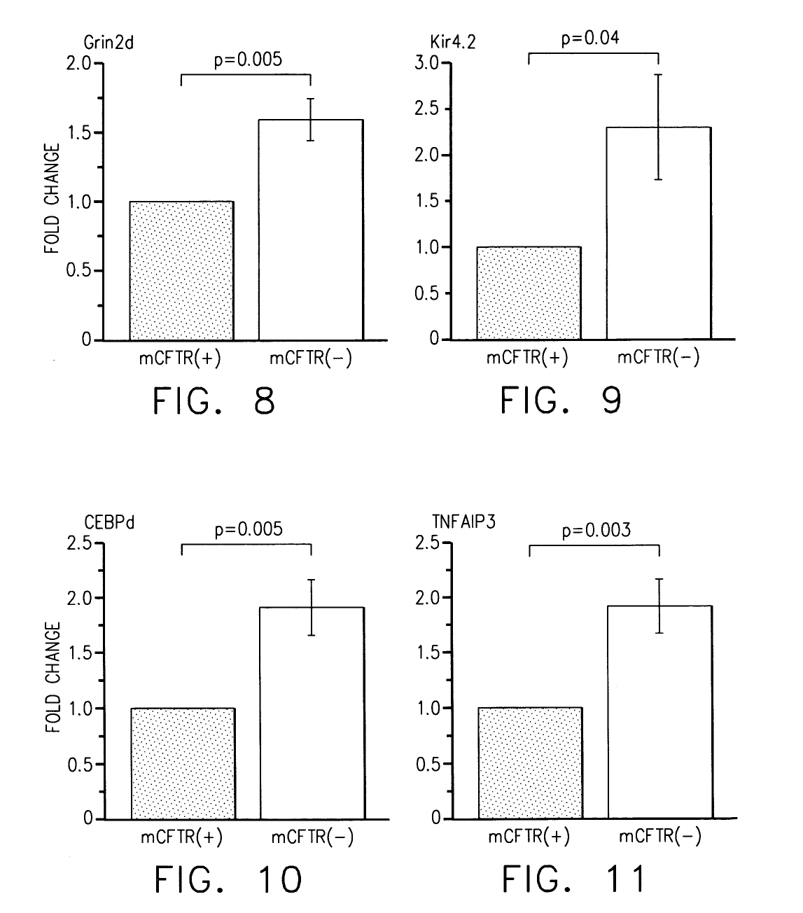

Changes in selected RNA levels identified in the microarray analysis are validated by RT-PCR. mRNA levels are normalized using β-actin or GAPDH. Kir 4.2 (Kcnj15), CEBPδ, TNF-AIP-3 and Grin2d, mRNA are significantly increased in CFTR (−/−) mice compared to control littermates. See FIGS. 8, 9, 10 and 11. As expected, murine CFTR is not detectable by RT-PCR in mCFTR (−/−) mice, nor is hCFTR mRNA detected in lung from the iFABP-hCFTR bearing mice.

### EXAMPLE 3

Selected genes are subjected to intensive search to identify biological function and associated regulatory pathways. A U74Av2 annotation database with system identifiers is constructed for all the array elements and their associated Genbank accession numbers. Gene description, functional categories, biological processes, molecular functions, cellular components, protein domain and literature information are identified. Information resources include NetAffy (http://www.affymetrix.com), Source Search (http://genomewww5.stanford.edu/cgi-bin/SMD/source/), BLAST NCBI, Locus Link, mouse-human homolog search (http://www.ncbi.nlm.nih.gov), and Gene Ontology Database (http://www.godatabase.org/chi-bin/go.cgi). Differentially expressed genes are classified into functional categories based on the gene ontology definition. To determine which functional category is over-represented in the selected gene list, the binomial probability is calculated for each category using entire U74Av2 (contains 12488 mice genes) as a reference dataset. The binomial probability is defined by the equation:

$$P ( k , n , p ) = {\sum }_{k = 0}^{n} {(}_{k}^{n} ) {{p}^{k} ( 1 - p )}^{n - k}$$

It returns the probability of getting k successes out of n trials if the probability is p in the given population (U74Av2). Potential protein/protein or protein/DNA interactions, are identified using the published literature information.

Analysis of arrays is prepared from pairs of mCFTR (−/−) mice and mCFTR (+/+) littermates (those lacking the iFABP-hCFTR transgene), confirm the microarray findings, demonstrating both the lack of mCFTR mRNA in lungs of the mCFTR (−/−) and that RNA changes are independent of the iFABP-hCFTR transgene.

### EXAMPLE 4

Lungs from postnatal animals are inflation fixed with 4% paraformaldehyde at 25 cm H₂O pressure via a tracheal cannula. Lung tissue is processed according to standard methods and embedded in paraffin. Procedures for immunostaining are described in Whitsett et al, J. Biol. Chem., (2002) 277:22743-49. Rabbit monoclonal antibody against the 110 kDa Mac-3 antigen is used at 1:40,000 to identify alveolar macrophages (Pharmingen, San Diego, Calif.).

Results:

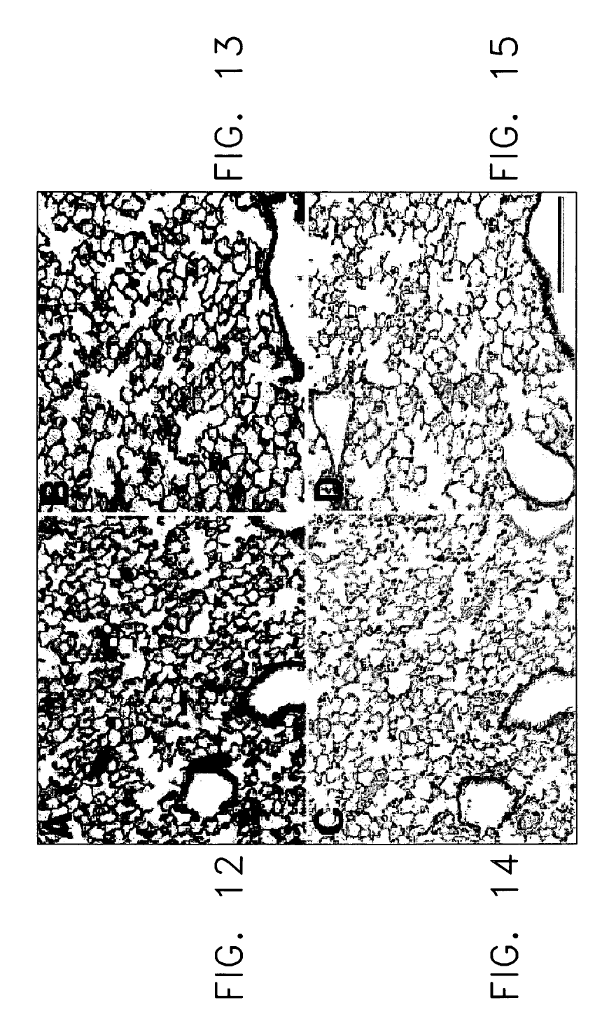

Lung histology in adult iFABP-hCFTR, CFTR (−/−); iFABP-hCFTR, CFTR (+/+), and CFTR(+/+) control mice is not different. See FIGS. 11, 12, 13 and 14. There is no evidence of pulmonary inflammation, infection or remodeling. Mac-3 staining is used to identify alveolar macrophages. Numbers and histology of alveolar macrophages are not altered by mCFTR. Histologic analyses demonstrate no structural abnormalities, infection or inflammation in the lungs of these mice, supporting the concept that changes in gene expression are related to CFTR and not to age or lung disease.

Maintenance of pulmonary homeostasis in the mCFTR (−/−) mouse is associated with complex adaptive responses in gene expression. CFTR influences RNAs encoding transcription factors, ion channels, membrane receptors, cytokines, and intracellular trafficking proteins. Finally, CFTR alters the expression of a number of proteins that interact with CFTR via protein-protein interactions perhaps representing transcriptional responses to functions mediated by CFTR (−/−) protein complexes. The diversity of genes whose expression is altered by CFTR support the concept that, in addition to regulation of Cl-transport, CFTR plays diverse roles in multiple cellular functions. Pulmonary homeostasis in the CFTR (−/−) mouse is maintained by complex genomic responses to the lack of CFTR rather than by the action of a single alternative Cl-channel. Finally, the genes and pathways identified in this invention provide new links between CFTR and cellular processes that can influence the pathogenesis of CF lung disease.

While specific embodiments of the present invention have been described, it will be apparent to those skilled in the art that various modifications thereto can be made without departing from the spirit and scope of the present invention as defined in the appended claims.

1. A method for treating cystic fibrosis in a subject having CF-affected cells, which comprises the step of: administering to the subject having CF-affected cells a therapeutically effective amount of a member selected from the group consisting of CFTR modifier polypeptides; genetic regulators for CFTR modifier genes; regulators for CFTR modifier polypeptides; and combinations thereof.

2. The method of claim 1 which comprises administering to the subject a CFTR modifier polypeptide from the group consisting of guanine nucleotide binding protein a subunit, membrane glycoprotein, ras-related dexamethasone inducible protein (DEXRAS1), ATP-sensitive inward rectifier potassium channel 14, zinc finger protein (Peg3), secreted frizzled related protein sFRP-2, and Connexin 37 Gap junction membrane channel protein alpha 4, and human hematopoietic specific protein 1 (GenBank accession number X84797); CFTR modifier polypeptides expressed by a CFTR modifier gene selected from the group consisting of Kir4.2, EST Affymetrix ID#92319, Repetin, SWAP-70, vq96e09.41, uo89c05.x1, Preproapelin, Caspase-12, islet cell autoantigen 1, Natriuretic peptide precursor type A, U[-M-BH1-ang-b-04-0-U].s1, C88243, U[-M-BH0-ajq-h-03-0-U].s1, Butyrylcholinesterase, homeo box A5, Wnt10a, Cystic fibrosis transmembrane conductance regulator homologue, and those CFTR modifier genes listed in Tables 1 and 2; a genetic regulator for a CFTR modifier gene selected from the group consisting of those expressing guanine nucleotide binding protein a subunit, membrane glycoprotein, ras-related dexamethasone inducible protein (DEXRAS1), ATP-sensitive inward rectifier potassium channel 14, zinc finger protein (Peg3), secreted frizzled related protein sFRP-2, Connexin 37 Gap junction membrane channel protein alpha 4, and human hematopoietic specific protein 1 (GenBank accession number X84797), and CFTR modifier polypeptides expressed by a CFTR modifier gene selected from the group consisting of Kir4.2, EST Affymetrix ID#92319, Repetin, SWAP-70, vq96e09.41, uo89c05.x1, Preproapelin, Caspase-12, islet cell autoantigen 1, Natriuretic peptide precursor type A, U[-M-BH1-ang-b-04-0-U].s1, C88243, U[-M-BH0-ajq-h-03-0-U].s1, Butyrylcholinesterase, homeo box A5, Wnt10a, Cystic fibrosis transmembrane conductance regulator homologue, and those CFTR modifier genes listed in Tables 1 and 2; a regulator for a CFTR modifier polypeptide selected guanine nucleotide binding protein α subunit, membrane glycoprotein, ras-related dexamethasone inducible protein (DEXRAS1), ATP-sensitive inward rectifier potassium channel 14, zinc finger protein (Peg3), secreted frizzled related protein sFRP-2, Connexin 37 Gap junction membrane channel protein alpha 4, and human hematopoietic specific protein 1 (GenBank accession number X84797), CFTR modifier polypeptides expressed by a CFTR modifier gene selected from the group consisting of Kir4.2, EST Affymetrix ID#92319, Repetin, SWAP-70, vq96e09.41, uo89c05.x1, Preproapelin, Caspase-12, islet cell autoantigen 1, Natriuretic peptide precursor type A, mSox7, U[-M-BH1-ang-b-04-0-U].s1, C88243, U[-M-BH0-ajq-h-03-0-U].s1, Butyrylcholinesterase, homeo box A5, Wnt10a, Cystic fibrosis transmembrane conductance regulator homologue, and those CFTR modifier genes listed in Tables 1 and 2, or combinations thereof.

3. The method of claim 2 which comprises administering to the subject a CFTR modifier polypeptide expressed by a CFTR modifier gene selected from the group consisting of Kir4.2; potassium inwardly-rectifying channel, member 15; subfamily J, member 15; solute carrier family 38, member 4, proteasome (prosome, macropain) 26S subunit, ATPase 3; and glutamate receptor, ionotrpic, NMDA2D (epilson 4).

4. The method of claim 2 which comprises administering to the subject a genetic regulator for a CFTR modifier gene selected from group consisting of Kir4.2; potassium inwardly-rectifying channel, member 15; subfamily J, member 15; solute carrier family 38, member 4, proteasome (prosome, macropain) 26S subunit, ATPase 3; and glutamate receptor, ionotrpic, NMDA2D (epilson 4), cystic fibrosis transmembrane conductance regulator homolog and mouse gap junction gene connexin 37.

5. The method of claim 2 which comprises administering to the subject a regulator for a CFTR modifier polypeptide expressed by a CFTR modifier gene selected from group consisting of Kir4.2; potassium inwardly-rectifying channel, member 15; subfamily J, member 15; solute carrier family 38, member 4, proteasome (prosome, macropain) 26S subunit, ATPase 3; and glutamate receptor, ionotrpic, NMDA2D (epilson 4), cystic fibrosis transmembrane conductance regulator homolog and mouse gap junction gene connexin 37.

6. The method of claim 2 which comprises administering to the subject a CFTR modifier polypeptide expressed by the Kir4.2 gene; a genetic regulator for the Kir4.2 gene; or a regulator for the CFTR modifier polypeptide expressed by the Kir4.2 gene.

7. A method for detecting and/or identifying a member selected from the group consisting of CFTR modifier genes, CFTR modifier polypeptides, genetic regulators for CFTR modifier genes, regulators for CFTR modifier polypeptides and combinations thereof, which comprises the step of: contacting a sample containing a potential CFTR modifier gene, CFTR modifier polypeptide, genetic regulator of a CFTR modifier gene, regulator of a CFTR modifier polypeptide or combination thereof; with an indicator that identifies when a potential CFTR modifier gene, CFTR modifier polypeptide, genetic regulator of CFTR modifier genes, regulator of CFTR modifier polypeptides, or combinations thereof is present in the sample.

8. The method of claim 7 which comprises contacting a sample containing a potential CFTR modifier gene or CFTR modifier polypeptide with the indicator.

9. A method for detecting and identifying a CFTR modifier gene which comprises the steps of:
a. providing a CFTR mutant mouse or a mouse where CFTR is absent;
b. isolating from the CFTR mutant mouse genetic material that encodes the CFTR mutant polypeptide or genetic material from the mouse where CFTR is absent that does not encode CFTR; and
c. using the isolated genetic material to identify changes in gene expression that compensate for the mutant CFTR or the absence of CFTR.

10. A method for detecting and identifying a potential CFTR modifier gene in a human suspected of having a CFTR mutation or in which CFTR is potentially absent, which comprises the steps of:
a. isolating from the suspected human genetic material that potentially encodes the CFTR mutant polypeptide or genetic material from which CFTR is potentially absent that does not encode CFTR; and
b. using the isolated genetic material to identify any potential changes in gene expression associated with cystic fibrosis or the absence of CFTR.

11. An array for screening for potential CFTR modifier genes, CFTR modifier polypeptides, genetic regulators of CFTR modifier genes, regulators of CFTR modifier polypeptides, and combinations thereof, the array comprising a substrate with a plurality of biological materials attached to the surface in discrete regions, the biological materials being capable of identifying potential CFTR modifier genes, CFTR modifier polypeptides, genetic regulators of CFTR modifier genes, regulators of CFTR modifier polypeptides or combinations thereof.

12. The array of claim 11 wherein the biological materials are CFTR modifier genes selected from the group consisting of CFTR modifier genes expressing guanine nucleotide binding protein a subunit, membrane glycoprotein, ras-related dexamethasone inducible protein (DEXRAS1), ATP-sensitive inward rectifier potassium channel 14, zinc finger protein (Peg3), secreted frizzled related protein sFRP-2, Connexin 37 Gap junction membrane channel protein alpha 4, and human hematopoietic specific protein 1 (GenBank accession number X84797); CFTR modifier genes selected from the group consisting of Kir4.2, EST Affymetrix ID#92319, Repetin, SWAP-70, vq96e09.41, uo89c05.x1, Preproapelin, Caspase-12, islet cell autoantigen 1, Natriuretic peptide precursor type A, mSox7, U[-M-BH1-ang-b-04-0-U].s1, C88243, U[-M-BH0-ajq-h-03-0-U].s1, Butyrylcholinesterase, homeo box A5, Wnt10a, Cystic fibrosis transmembrane conductance regulator homologue, CFTR modifier genes listed in Tables 1 and 2; or CFTR modifier polypeptides expressed by any of these CFTR modifier genes; and combinations thereof.

13. The array of claim 12 wherein the biological materials are the CFTR modifier genes.

14. The array of claim 13 wherein the biological materials are the CFTR modifier genes selected from the group consisting of those expressing guanine nucleotide binding protein a subunit, membrane glycoprotein, ras-related dexamethasone inducible protein (DEXRAS1), ATP-sensitive inward rectifier potassium channel 14; Kir4.2, EST Affymetrix ID#92319, Repetin, SWAP-70, vq96e09.41, and uo89c05.x1, CFTR modifier genes listed in Table 1, and combinations thereof.

15. The array of claim 13 wherein the biological materials are the CFTR modifier genes selected from the group consisting of those expressing secreted frizzled related protein sFRP-2, Connexin 37 Gap junction membrane channel protein alpha 4, and human hematopoietic specific protein 1 (GenBank accession number X84797); Preproapelin, Caspase-12, islet cell autoantigen 1, Natriuretic peptide precursor type A, mSox7, U[-M-BH1-ang-b-04-0-U].s1, C88243, U[-M-BH0-ajq-h-03-0-U].s1, Butyrylcholinesterase, homeo box A5, Wnt10a, Cystic fibrosis transmembrane conductance regulator homologue, CFTR modifier genes listed in Table 2, and combinations thereof.

16. A method for screening for potential CFTR modifier genes, CFTR modifier polypeptides, genetic regulators of CFTR modifier genes, regulators of CFTR modifier polypeptides, or combinations thereof, the method comprising the step of: contacting the array of claim 10 with a sample containing a potential CFTR modifier gene, CFTR modifier polypeptide, genetic regulator of CFTR modifier gene, regulator of a CFTR modifier polypeptide, or combination thereof, the array having associated therewith an indicator for identifying if a potential CFTR modifier gene, CFTR modifier polypeptide, genetic regulator of CFTR modifier genes, regulator of CFTR modifier polypeptide, or combination thereof is present in the sample.

17. A method for screening for a potential genetic regulator of a CFTR modifier gene, which comprises the step of: contacting a sample containing a potential genetic regulator of a CFTR modifier gene with a cell culture transfected with a reporter gene capable of identifying a genetic regulator of a CFTR modifier gene.

18. The method of claim 17 which comprises the step of contacting a sample wherein the reporter gene is capable of identifying a genetic regulator of a CFTR modifier gene selected from the group consisting of CFTR modifier genes expressing guanine nucleotide binding protein a subunit, membrane glycoprotein, ras-related dexamethasone inducible protein (DEXRAS1), ATP-sensitive inward rectifier potassium channel 14, zinc finger protein (Peg3), secreted frizzled related protein sFRP-2, Connexin 37 Gap junction membrane channel protein alpha 4, and human hematopoietic specific protein 1 (GenBank accession number X84797); CFTR modifier genes selected from the group consisting of Kir4.2, EST Affymetrix ID#92319, Repetin, SWAP-70, vq96e09.41, uo89c05.x1, Preproapelin, Caspase-12, islet cell autoantigen 1, Natriuretic peptide precursor type A, mSox7, U[-M-BH1-ang-b-04-0-U].s1, C88243, U[-M-BH0-ajq-h-03-0-U].s1, Butyrylcholinesterase, homeo box A5, Wnt10a, Cystic fibrosis transmembrane conductance regulator homologue, and CFTR modifier genes listed in Tables 1 and 2; or CFTR modifier polypeptides expressed by any of these CFTR modifier genes; and combinations thereof.

19. The method of claim 18 which comprises the step of contacting a sample wherein the reporter gene is capable of identifying a genetic regulator of a CFTR modifier gene selected from the group consisting of CFTR modifier genes expressing guanine nucleotide binding protein a subunit, membrane glycoprotein, ras-related dexamethasone inducible protein (DEXRAS1), ATP-sensitive inward rectifier potassium channel 14 ATP-sensitive inward rectifier potassium channel 14; Kir4.2, EST Affymetrix ID#92319, Repetin, SWAP-70, vq96e09.41, and uo89c05.x1, CFTR modifier genes listed in Table 1, and combinations thereof.

20. The method of claim 18 which comprises the step of contacting a sample wherein the reporter gene is capable of identifying a genetic regulator of a CFTR modifier gene selected from the group consisting of those expressing secreted frizzled related protein sFRP-2, Connexin 37 Gap junction membrane channel protein alpha 4, and human hematopoietic specific protein 1 (GenBank accession number X84797); Preproapelin, Caspase-12, islet cell autoantigen 1, Natriuretic peptide precursor type A, mSox7, U[-M-BH1-ang-b-04-0-U].s1, C88243, U[-M-BH0-ajq-h-03-0-U].s1, Butyrylcholinesterase, homeo box A5, Wnt10a, Cystic fibrosis transmembrane conductance regulator homologue, CFTR modifier genes listed in Table 2, and combinations thereof.

21. A method for screening for a potential regulator of a CFTR modifier polypeptide, which comprises the step of: contacting a sample containing a potential CFTR modifier polypeptide with a cell culture transfected with an expression vector comprising the CFTR modifier gene that is capable of identifying a CFTR modifier polypeptide.

22. The method of claim 21 which comprises the step of contacting a sample wherein the expression vector comprises a CFTR modifier gene selected from the group consisting of those expressing guanine nucleotide binding protein a subunit, membrane glycoprotein, ras-related dexamethasone inducible protein (DEXRAS1), ATP-sensitive inward rectifier potassium channel 14, zinc finger protein (Peg3), secreted frizzled related protein sFRP-2, Connexin 37 Gap junction membrane channel protein alpha 4, and human hematopoietic specific protein 1 (GenBank accession number X84797); CFTR modifier genes selected from the group consisting of Kir4.2, EST Affymetrix ID#92319, Repetin, SWAP-70, vq96e09.41, uo89c05.x1, Preproapelin, Caspase-12, islet cell autoantigen 1, Natriuretic peptide precursor type A, mSox7, U[-M-BH1-ang-b-04-0-U].s1, C88243, U[-M-BH0-ajq-h-03-0-U].s1, Butyrylcholinesterase, homeo box A5, Wnt10a, Cystic fibrosis transmembrane conductance regulator homologue, CFTR modifier genes listed in Tables 1 and 2; or CFTR modifier polypeptides expressed by any of these CFTR modifier genes; and combinations thereof.

23. The method of claim 22 which comprises the step of contacting a sample wherein the expression vector comprises a CFTR modifier gene selected from the group consisting of those expressing guanine nucleotide binding protein a subunit, membrane glycoprotein, ras-related dexamethasone inducible protein (DEXRAS1), ATP-sensitive inward rectifier potassium channel 14; Kir4.2, EST Affymetrix ID#92319, Repetin, SWAP-70 vq96e09.41, and uo89c05.x1, CFTR modifier genes listed in Table 1, and combinations thereof.

24. The method of claim 22 which comprises the step of contacting a sample wherein the expression vector comprises a CFTR modifier gene selected from the group consisting of those expressing secreted frizzled related protein sFRP-2, Connexin 37 Gap junction membrane channel protein alpha 4, and human hematopoietic specific protein I (GenBank accession number X84797); Preproapelin, Caspase-12, islet cell autoantigen 1, Natriuretic peptide precursor type A, mSox7, U[-M-BH1-ang-b-04-0-U].s1, C88243, U[-M-BH0-ajq-h-03-0-U].s1, Butyrylcholinesterase, homeo box A5, Wnt10a, Cystic fibrosis transmembrane conductance regulator homologue, CFTR modifier genes listed in Table 2, and combinations thereof.

25. A method for treating cystic fibrosis in a subject having CF-affected cells, which comprises the step of: administering to the subject having CF-affected cells a therapeutically effective amount of a genetic regulator for CFTR modifier gene.

26. The method of claim 25 which comprises administrating a genetic regulator selected from the group consisting of transcription factors, proto-oncogenes which enhance transcription, interferon gamma and analogues thereof, NF-κB and analogues thereof, nuclear factor of activated cells, calcium channel activating agents, ets factor agents, GM-CSF; IL-6, IL-1α, IL-1β, INF-γ and analogues thereof, cAMP analogues, activators of adenylate cyclase, cAMP phosphodiesterase inhibitors, retinoids, orphan receptor activators; retinoic acid receptor agonists, retinols, retinoic acid and analogues thereof; steriodogenic factor, glucocortiods, glucocorticoid analogues, mineralcorticoids, estrogens, progestins, and analogues thereof; betamethasone, Decadron, and mixtures thereof.

27. The method of claim 26 wherein the genetic regulator is administered to a mammal.

28. The method of claim 27 wherein the genetic regulator is administered to a human.

29. A method for treating cystic fibrosis in a subject having CF-affected cells, which comprises the step of: administering to the subject having CF-affected cells a therapeutically effective amount of a polypeptide regulator for CFTR modifier polypeptide.

30. The method of claim 29 which comprises administrating a polypeptide regulator selected from the group consisting of agents that activate adenylate cyclase in target cells, cAMP agonists, cAMP supplements, polypeptide hormones that stimulate cAMP, cAMP phosphodiesterase inhibitors that block cAMP breakdown,; cAMP-specific inhibitors, glucocorticoid, TGF-β (SMAD₃); potassium K_{ATP} channel openers, potassium BK_{Ca} channel openers, benzimidazolones, UTP, 8-methoxypsoralen, and genistein, calcium ion agonists, human DNase 1 sodium channel blockers, pancreatic enzyme supplements; and mixtures thereof.

31. The method of claim 30 which comprises administrating a polypeptide regulator selected from the group consisting of forskolin, isoproterenol and albuterol, cAMP and analogues thereof, vasopressin, alkylxanthines, aminophylline; Rolipram, glucocorticoid, TGF-, (SMAD₃), cromakalim, pinacidil, nicorandil, minoxidil sulphate, aprikalim, diazoxide, NS004, 1-ethyl-2-benzimidazolinone, fenamates, dehydroxoyasaponin-I, maxikdiol, cromakalim, nirendipine, and phloretin, UTP, 8-methoxypsoralen, genistein; ionomycin, A23187, carbachol, bradykinin, duramycin, thapsigargin, human DNase 1, amiloride, triamterene, pancreatic enzyme supplements, and mixtures thereof.

32 The method of claim 30 which comprises administrating a polypeptide regulator selected from the group consisting of alkylxanthine selected from the group consisting of 3-isobutyl-1-methylxanthine, 1,3-dimethylxanthine, papaverine, pentoxifilline, caffeine, and mixtures thereof, and benzimidazole or benzimadazole derivatives selected from the group consisting of omeprazole, lansoprazole, thimoprazole, pantoprazole4-trifluoromethyl-2-[(4-methoxy-2-pyridylmethyl)thiol]-(1H)-benzimidazole; 4-trifluoromethyl-2-[(4-methoxy-3-methyl-2-pyridylmethyl)thio]-(1H)-benzimidazole; 4-trifluoromethyl-2-[(4-methoxy-5-methyl-2-pyridylmethyl)thio]-(1H)-benzimidazole; 4-trifluoromethyl-2-[(4-methoxy-3,5-dimethyl-2-pyridylmethyl)thio]-(1H)-benzimidazole; 5-trifluoromethyl-2-[(4-methoxy-2-pyridylmethyl)thio]-(1H)-benzimidazole; 5-trifluoromethyl-2-[(4-methoxy-3-methyl-2-pyridylmethyl)thio]-(1H)-benzimidazole; 5-trifluoromethyl-2-[(4-methoxy-5-methyl-2-pyridylmethyl)thio]-(1H)-benzimidazole; 5-trifluoromethyl-2-[(4-methoxy-3,5-dimethyl-2-pyridylmethyl)thio]-(1H)-benzimidazole; 4-trifluoromethyl-2-[(4-methoxy-2-pyridylmethyl)-sulfinyl]-(1H)-benzimidazole; 4-trifluoromethyl-2-[(4-methoxy-3-methyl-2-pyridylmethyl)sulfinyl]-(1H)-benzimidazole; 4-trifluoromethyl-2-[(4-methoxy-5-methyl-2-pyridylmethyl)sulfinyl]-(1H)-benzimidazole; 4-trifluoromethyl-2-[(4-methoxy-3,5-dimethyl-2-pyridylmethyl)sulfinyl]-(1H)-benzimidazole; 5-trifluoromethyl-2-[(4-methoxy-2-pyridylmethyl)sulfinyl]-(1H)-benzimidazole; 5-trifluoromethyl-2-[(4-methoxy-3-methyl-2-pyridylmethyl)sulfinyl]-(1H)-benzimidazole; 5-trifluoromethyl-2-[(4-methoxy-5-methyl-2-pyridylmethyl)sulfinyl]-(1H)-benzimidazole and 5-trifluoromethyl-2-[(4-methoxy-3,5-dimethyl-2-pyridylmethyl)sulfinyl]-(1H)-benzimidazole; 2-[2-(4-methoxy)-pyridylmethylsulfinyl]-(5-acetyl-6-methyl)-benzimidazole; 2-[2-(4-methoxy)-pyridylmethylsulfinyl]-(4,6-dimethyl)-benzimidazole; 2-[2-(3,5-dimethyl-4-methoxy)-pyridylmethylsulfinyl]-(5-acetyl-6-methyl)-benzimidazole; 2-[2-(4-methoxy)-pyridylmethylsulfinyl]-(5-carbomethoxy-6-methyl)-benzimidazole; 2-[2-(4-ethoxy)-pyridylmethylsulfinyl]-(5-carbomethoxy-6-methyl)-benzimidazole; 2-[2-(3-methyl-4-methoxy)-pyridylmethylsulfinyl]-(5-carbomethoxy-6-methyl)-benzimidazole; 2-[2-(3,5-dimethyl-4-methoxy)-pyridylmethylsulfinyl]-(5-carbomethoxy-6-methyl)-benzimidazole; 2-[2-(4-methoxy-5-methyl)-pyridylmethylsulfinyl]-(5-carbomethoxy)-benzimidazole; 2-[2-(3,5-dimethyl-4-methoxy)-pyridylmethylsulfinyl]-5-carbomethoxy)-benzimidazole; 2-[2-(3,5-dimethyl-4-methoxy)-pyridylmethylsulfinyl]-(5-acetyl)-benzimidazole; 2-[2-(4-methoxy-5-methoxy)-pyridylmethylsulfinyl]-(5-methoxy)-benzimidazole; 2-[2-(3,5-dimethyl-4-methoxy)-pyridylmethylsulfinyl]-(5-methoxy)-benzimidazole; 2-[2-(3,5-dimethyl-4-methoxy)-pyridylmethylsulfinyl]-(5-methyl)-benzimidazole; 2-[2-(3,5-dimethyl-4-methoxy)-pyrdylmethysulfinyl]-benzimidazole; 2-[2-(3,5-dimethyl-4-methoxy)-pyridylmethylsulfinyl]-(5-chloro)-benzimidazole; 2-[2-[3-methyl-4-(2,2,2-trifluoroethoxy)pyridyl]methylsulfinyl]benzimidazole(lansoprazole); 2-[2-[3-methyl-4-(2,2,3,3-tetrafluoropropoxy)pyridyl]methylthio]benzimidazole; 2-[(2-pyridyl)methylsulfinyl]benzimidazole(thimoprazole); 2-[2-(3,5-dimethyl-4-methoxypyridyl)methylsulfinyl]-5-methoxy-1H-benzimidazole (omeprazole); 2-[2-[4-(3-methoxypropoxy)-3-methylpyridyl]methylsulfinyl]-1H-benzimidazole; 2-[2-(3,4-dimethoxypyridyl)methylsulfinyl]-5-difluoromethoxy-1H-benzimidazole (pantoprazole); 4-methyl-3-(2,2,2-trifluoroethoxy)-5H-pyrido[1′,2′:4,5]1,2,4]thiaziano[2,3-a]benzimidazole-13-ium tetrafluoroborate, pharmaceutically acceptable salt thereof, and mixtures thereof.

33. The method of claim 32 wherein the polypeptide regulator is administered to a mammal.

34. The method of claim 33 wherein the polypeptide regulator is administered to a human.

35. The method of claim 32 which comprises administrating a polypeptide regulator selected from the group consisting of forskolin and 3-isobutyl-1-methylxanthine.


SEQUENCE LISTING (8 sequences)

SEQ ID NO: 1 - DNA, 1443 bp, Mouse
gccctctacatgcatgctcgagcggccgccagtgtgatggatatctgcagaattcggcttggtaagccaggtgggagaaggggagcgaggacgcgccacttaccctgcagaagattcctgacctacagagtgagtgaccaggtggtccaaagatggatgccattcaccttggcatgtccagtgccccactggtgaagcataccaacggggttggactcaaggcccacagaccccgagtcatgtcaaagggtgggcacagtaatgtgagaatcgataaggtagacggaatctatttactctacctccaggacttgtggacaaccgtcatcgacatgaagtggcgatacaagctcaccctatttgctgccacctttgtgatgacctggtttctgtttggagtggtctactatgccatagcctttattcatggtgacttacaacttggggaatctaattccaaccacacaccctgcattatgaaagtggactctctcacgggagcattcctcttttccttggaatctcagacaaccattggctacggggtccgttccatcacagaggagtgtccccatgctatcttcctcttagtcgcccaactggtcatcaccacattgattgagatcttcattacggggacctttctggctaaaattgcaagacccaaaaagcgagccgagaccattaagttcagccactgtgctgtcatcagcaagcagaatggaaagctatgcctggtcatcagggtggccgacatgaggaagagtctcctgattcagtgccagctctctggaaaactcctgcagacacacgtcaccaaagagggagaacgcattctcgtcaaccaggccactgtcaaattccacgtggactcctcttccgagagtcccttcctcatcctgcccatgaccttctaccacgtgttggatgagacaagccccctgcgggacctcacaccccaaaacgtaaaggagaaggagtttgagctggtggtacttctcaacgccacggtggagtctaccagcgccgtctgccagagccgaacgtcttacatcccggaggagatctactggggctttgagtttgtgcctgtggtttctctctccaaaaatggaaagtatgtggctgatttcagtcaatttgagcagatcaggaagagcccgggttgtaccttctactgtgccgattctgagaagcagaagcttgaagaacagtacaggcaagaggaccagagggagcgggagctgaggagcctcctgctacagcagagcaatgtctgactccaccggcctctgggcgtctcctaaccagggctcctttcaaaagcaatggctgccatgaacgtgggatgggtggccccacgtaccaaatcacacagacgttaagccgaattccagcacactggcggccgttactagtggatccgagctcggtaccaagcca

SEQ ID NO: 2 - protein, 375 aa, Mouse
MDAIHLGMSSAPLVKHTNGVGLKAHRPRVMSKGGHSNVRIDKVDGIYLLYLQDLWTTVIDMKWRYKLTLFAATFVMTWFLFGVVYYAIAFIHGDLQLGESNSNHTPCIMKVDSLTGAFLFSLESQTTIGYGVRSITEECPHAIFLLVAQLVITTLIEIFITGTFLAKIARPKKRAETIKFSHCAVISKQNGKLCLVIRVADMRKSLLIQCQLSGKLLQTHVTKEGERILVNQATVKFHVDSSSESPFLILPMTFYHVLDETSPLRDLTPQNVKEKEFELVVLLNATVESTSAVCQSRTSYIPEEIYWGFEFVPVVSLSKNGKYVADFSQFEQIRKSPGCTFYCADSEKQKLEEQYRQEDQRERELRSLLLQQSNV

SEQ ID NO: 3 - DNA, 2732 bp, Human
cagatctctgggatcttcagctgagattgggccccagcacctggactcaaggcagtagcagaatcccatggtagccaggtgggtgaaggggagcgaggacgttctacctgccttgaagaagacacctgacctgcggagtgagtgaccagtgtttccagagcctggcaatggatgccattcacatcggcatgtccagcacccccctggtgaagcacactgctggggctgggctcaaggccaacagaccccgcgtcatgtccaagagtgggcacagcaacgtgagaattgacaaagtggatggcatatacctactctacctgcaagacctgtggaccacagttatcgacatgaagtggagatacaaactcaccctgttcgctgccacttttgtgatgacctggttcctttttggagtcatctactatgccatcgcgtttattcatggggacttagaacccggtgagcccatttcaaatcataccccctgcatcatgaaagtggactctctcactggggcgtttctcttttccctggaatcccagacaaccattggctatggagtccgttccatcacagaggaatgtcctcatgccatcttcctgttggttgctcagttggtcatcacgaccttgattgagatcttcatcaccggaaccttcctggccaaaatcgccagacccaaaaagcgggctgagaccatcaagttcagccactgtgcagtcatcaccaagcagaatgggaagctgtgcttggtgattcaggtagccaatatgaggaagagcctcttgattcagtgccagctctctggcaagctcctgcagacccacgtcaccaaggagggggagcggattctcctcaaccaagccactgtcaaattccacgtggactcctcctctgagagccccttcctcattctgcccatgacattctaccatgtgctggatgagacgagccccctgagagacctcacaccccaaaacctaaaggagaaggagtttgagcttgtggtcctcctcaatgccactgtggaatccaccagcgctgtctgccagagccgaacatcttatatcccagaggaaatctactggggttttgagtttgtgcctgtggtatctctctccaaaaatggaaaatatgtggctgatttcagtcagtttgaacagattcggaaaagcccagattgcacattttactgtgcagattctgagaaacagcaactcgaggagaagtacaggcaggaggatcagagggaaagagaactgaggacacttttattacaacagagcaatgtctgatcacaggggcgccatccaggtttaaccctgcaagctgtttccacatcagaactcccttcaaacacaaagattgctgtgaaaacgaaaatgtgtagacgcactctcaaaaactgcacggacatacaaaatcaatcttttcctttgatcttgtggctaaaccagcatttctgtgtttgagagatttcctgttaggtgcttcgtctgaaagtgaactctcataattcaaattgtataaaataaagctacatttctaagagcttggtgtagggcaattggaataatgtcctgttagataaacagacatttagcaatcctgacattaaaaggaaatgtatttctatacaagattattagctgtaatacaagatatttatttaaccaatgaccttatggctgagagttgaattgtggttcagtattcattgatctcactgctttaaaacatgctctttttgttcaagcaggaaaaatattatatctaattatgtctatgtggtaatacctaaaaagaggtagagagactctatgttcacaaaatatagtaatttcatttttacctttcttcagtggactgaattgtatggaaggaaattgatcagatctgtaattcacaactgtggaaacctacacaaatggggctgatgccattgtttcctctattttattttatttatttttgaatccactatctctttccattcaaaatatttccattcaaaatgtggttcacagacccgccacatcagcaccatccgagagcttattagtaatgtgtagattctcatgtcccagccagacttacagaattggggtctgtatcttaacaagaaccccagatgatttgtgtacaaatgaaagttcgaaagctgtgctctggacagtgttctcaaagcaactttctgaagccgtgtggaaaactgactctgtgtgggtcccgtatccctgtggcatccagacctggcctctcttcatttcattattattggccaatatttctttcagccatttcatttcttatctattaaaatggcttgcccataaaggaactgcataggattatcccctgacattccaagagcaaaataaagctcttgagagtaattgttgtctcagtcatgcttgctgattacagttagcacaaaagaaaaattcagctgcctgacaatacaggaaatgttctcagatgctgatgtttgtaaggtccggtggggccatgaggaagaagaggagctgaaggtaagaaactcataaacaagatgactctttgatgcatgaacaagatttgaaaatctcaagcctgtaaagaatacccctgctatttaaataaagctcataccaagaggtaacattttgccccgggccaaattcaggggtctagtgccctgcattcctttgaggcaaaaaataaatgggctatgactggttaaatgtccaaaa

SEQ ID NO: 4 - DNA, 2919 bp, Human
ctcctctgcccaatgtctcccaatctctttcctttctctcttcagttcctccaggtaattcttactcaaacttgtaccaacttgtttttgactgacagtgaacagtgagagagttttcttcattttgaggaaccctaaacacctatctttcccaaggcaacctgtctggactgagcatttctctgacttgacataacttcccatccagccaggagtctgcactcttcagtctttgcaggcagtagcagaatcccatggtagccaggtgggtgaaggggagcgaggacgttctacctgccttgaagaagacacctgacctgcggagtgagtgaccagtgtttccagagcctggcaatggatgccattcacatcggcatgtccagcacccccctggtgaagcacactgctggggctgggctcaaggccaacagaccccgcgtcatgtccaagagtgggcacagcaacgtgagaattgacaaagtggatggcatatacctactctacctgcaagacctgtggaccacagttatcgacatgaagtggagatacaaactcaccctgttcgctgccacttttgtgatgacctggttcctttttggagtcatctactatgccatcgcgtttattcatggggacttagaacccggtgagcccatttcaaatcataccccctgcatcatgaaagtggactctctcactggggcgtttctcttttccctggaatcccagacaaccattggctatggagtccgttccatcacagaggaatgtcctcatgccatcttcctgttggttgctcagttggtcatcacgaccttgattgagatcttcatcaccggaaccttcctggccaaaatcgccagacccaaaaagcgggctgagaccatcaagttcagccactgtgcagtcatcaccaagcagaatgggaagctgtgcttggtgattcaggtagccaatatgaggaagagcctcttgattcagtgccagctctctggcaagctcctgcagacccacgtcaccaaggagggggagcggattctcctcaaccaagccactgtcaaattccacgtggactcctcctctgagagccccttcctcattctgcccatgacattctaccatgtgctggatgagacgagccccctgagagacctcacaccccaaaacctaaaggagaaggagtttgagcttgtggtcctcctcaatgccactgtggaatccaccagcgctgtctgccagagccgaacatcttatatcccagaggaaatctactggggttttgagtttgtgcctgtggtatctctctccaaaaatggaaaatatgtggctgatttcagtcagtttgaacagattcggaaaagcccagattgcacattttactgtgcagattctgagaaacagcaactcgaggagaagtacaggcaggaggatcagagggaaagagaactgaggacacttttattacaacagagcaatgtctgatcacaggggcgccatccaggtttaaccctgcaagctgtttccacatcagaactcccttcaaacacaaagattgctgtgaaaacgaaaatgtgtagacgcactctcaaaaactgcacggacatacaaaatcaatcttttcctttgatcttgtggctaaaccagcatttctgtgtttgagagatttcctgttaggtgcttcgtctgaaagtgaactctcataattcaaattgtataaaataaagctacatttctaagagcttggtgtagggcaattggaataatgtcctgttagataaacagacatttagcaatcctgacattaaaaggaaatgtatttctatacaagattattagctgtaatacaagatatttatttaaccaatgaccttatggctgagagttgaattgtggttcagtattcattgatctcactgctttaaaacatgctctttttgttcaagcaggaaaaatattatatctaattatgtctatgtggtaatacctaaaaagaggtagagagactctatgttcacaaaatatagtaatttcatttttacctttcttcagtggactgaattgtatggaaggaaattgatcagatctgtaattcacaactgtggaaacctacacaaatggggctgatgccattgtttcctctattttattttatttatttttgaatccactatctctttccattcaaaatatttccattcaaaatgtggttcacagacccgccacatcagcaccatccgagagcttattagtaatgtgtagattctcatgtcccagccagacttacagaattggggtctgtatcttaacaagaaccccagatgatttgtgtacaaatgaaagttcgaaagctgtgctctggacagtgttctcaaagcaactttctgaagccgtgtggaaaactgactctgtgtgggtcccgtatccctgtggcatccagacctggcctctcttcatttcattattattggccaatatttctttcagccatttcatttcttatctattaaaatggcttgcccataaaggaactgcataggattatcccctgacattccaagagcaaaataaagctcttgagagtaattgttgtctcagtcatgcttgctgattacagttagcacaaaagaaaaattcagctgcctgacaatacaggaaatgttctcagatgctgatgtttgtaaggtccggtggggccatgaggaagaagaggagctgaaggtaagaaactcataaacaagatgactctttgatgcatgaacaagatttgaaaatctcaagcctgtaaagaatacccctgctatttaaataaagctcataccaagaggtaacattttgccccgggccaaattcaggggtctagtgccctgcattcctttgaggcaaaaaataaatgggctatgactggttaaatgtccaaaa

SEQ ID NO: 5 - DNA, 2867 bp, Human
ggtgtttggagagcccggactggatgcccactagaatcctgtgctgtgaacagtgagagagttttcttcattttgaggaaccctaaacacctatctttcccaaggcaacctgtctggactgagcatttctctgacttgacataacttcccatccagccaggagtctgcactcttcagtctttgcaggcagtagcagaatcccatggtagccaggtgggtgaaggggagcgaggacgttctacctgccttgaagaagacacctgacctgcggagtgagtgaccagtgtttccagagcctggcaatggatgccattcacatcggcatgtccagcacccccctggtgaagcacactgctggggctgggctcaaggccaacagaccccgcgtcatgtccaagagtgggcacagcaacgtgagaattgacaaagtggatggcatatacctactctacctgcaagacctgtggaccacagttatcgacatgaagtggagatacaaactcaccctgttcgctgccacttttgtgatgacctggttcctttttggagtcatctactatgccatcgcgtttattcatggggacttagaacccggtgagcccatttcaaatcataccccctgcatcatgaaagtggactctctcactggggcgtttctcttttccctggaatcccagacaaccattggctatggagtccgttccatcacagaggaatgtcctcatgccatcttcctgttggttgctcagttggtcatcacgaccttgattgagatcttcatcaccggaaccttcctggccaaaatcgccagacccaaaaagcgggctgagaccatcaagttcagccactgtgcagtcatcaccaagcagaatgggaagctgtgcttggtgattcaggtagccaatatgaggaagagcctcttgattcagtgccagctctctggcaagctcctgcagacccacgtcaccaaggagggggagcggattctcctcaaccaagccactgtcaaattccacgtggactcctcctctgagagccccttcctcattctgcccatgacattctaccatgtgctggatgagacgagccccctgagagacctcacaccccaaaacctaaaggagaaggagtttgagcttgtggtcctcctcaatgccactgtggaatccaccagcgctgtctgccagagccgaacatcttatatcccagaggaaatctactggggttttgagtttgtgcctgtggtatctctctccaaaaatggaaaatatgtggctgatttcagtcagtttgaacagattcggaaaagcccagattgcacattttactgtgcagattctgagaaacagcaactcgaggagaagtacaggcaggaggatcagagggaaagagaactgaggacacttttattacaacagagcaatgtctgatcacaggggcgccatccaggtttaaccctgcaagctgtttccacatcagaactcccttcaaacacaaagattgctgtgaaaacgaaaatgtgtagacgcactctcaaaaactgcacggacatacaaaatcaatcttttcctttgatcttgtggctaaaccagcatttctgtgtttgagagatttcctgttaggtgcttcgtctgaaagtgaactctcataattcaaattgtataaaataaagctacatttctaagagcttggtgtagggcaattggaataatgtcctgttagataaacagacatttagcaatcctgacattaaaaggaaatgtatttctatacaagattattagctgtaatacaagatatttatttaaccaatgaccttatggctgagagttgaattgtggttcagtattcattgatctcactgctttaaaacatgctctttttgttcaagcaggaaaaatattatatctaattatgtctatgtggtaatacctaaaaagaggtagagagactctatgttcacaaaatatagtaatttcatttttacctttcttcagtggactgaattgtatggaaggaaattgatcagatctgtaattcacaactgtggaaacctacacaaatggggctgatgccattgtttcctctattttattttatttatttttgaatccactatctctttccattcaaaatatttccattcaaaatgtggttcacagacccgccacatcagcaccatccgagagcttattagtaatgtgtagattctcatgtcccagccagacttacagaattggggtctgtatcttaacaagaaccccagatgatttgtgtacaaatgaaagttcgaaagctgtgctctggacagtgttctcaaagcaactttctgaagccgtgtggaaaactgactctgtgtgggtcccgtatccctgtggcatccagacctggcctctcttcatttcattattattggccaatatttctttcagccatttcatttcttatctattaaaatggcttgcccataaaggaactgcataggattatcccctgacattccaagagcaaaataaagctcttgagagtaattgttgtctcagtcatgcttgctgattacagttagcacaaaagaaaaattcagctgcctgacaatacaggaaatgttctcagatgctgatgtttgtaaggtccggtggggccatgaggaagaagaggagctgaaggtaagaaactcataaacaagatgactctttgatgcatgaacaagatttgaaaatctcaagcctgtaaagaatacccctgctatttaaataaagctcataccaagaggtaacattttgccccgggccaaattcaggggtctagtgccctgcattcctttgaggcaaaaaataaatgggctatgactggttaaatgtccaaaa

SEQ ID NO: 6 - protein, 375 aa, Human
MDAIHIGMSSTPLVKHTAGAGLKANRPRVMSKSGHSNVRIDKVDGIYLLYLQDLWTTVIDMKWRYKLTLFAATFVMTWFLFGVIYYAIAFIHGDLEPGEPISNHTPCIMKVDSLTGAFLFSLESQTTIGYGVRSITEECPHAIFLLVAQLVITTLIEIFITGTFLAKIARPKKRAETIKFSHCAVITKQNGKLCLVIQVANMRKSLLIQCQLSGKLLQTHVTKEGERILLNQATVKFHVDSSSESPFLILPMTFYHVLDETSPLRDLTPQNLKEKEFELVVLLNATVESTSAVCQSRTSYIPEEIYWGFEFVPVVSLSKNGKYVADFSQFEQIRKSPDCTFYCADSEKQQLEEKYRQEDQRERELRTLLLQQSNV

SEQ ID NO: 7 - DNA, 1708 bp, Human
gcagtagcagaatcccatggtagccaggtgggtgaaggggagcgaggacgttctacctgccttgaagaagacacctgacctgcggagtgagtgaccagtgtttccagagcctggcaatggatgccattcacatcggcatgtccagcacccccctggtgaagcacactgctggggctgggctcaaggccaacagaccccgcgtcatgtccaagagtgggcacagcaacgtgagaattgacaaagtggatggcatatacctactctacctgcaagacctgtggaccacagttatcgacatgaagtggagatacaaactcaccctgttcgctgccacttttgtgatgacctggttcctttttggagtcatctactatgccatcgcgtttattcatggggacttagaacccggtgagcccatttcaaatcataccccctgcatcatgaaagtggactctctcactggggcgtttctcttttccctggaatcccagacaaccattggctatggagtccgttccatcacagaggaatgtcctcatgccatcttcctgttggttgctcagttggtcatcacgaccttgattgagatcttcatcaccggaaccttcctggccaaaatcgccagacccaaaaagcgggctgagaccatcaagttcagccactgtgcagtcatcaccaagcagaatgggaagctgtgcttggtgattcaggtagccaatatgaggaagagcctcttgattcagtgccagctctctggcaagctcctgcagacccacgtcaccaaggagggggagcggattctcctcaaccaagccactgccaaattccacgtggactcctcctctgagagccccttcctcattctgcccatgacattctaccatgtgctggatgagacgagccccctgagagacctcacaccccaaaacctaaaggagaaggagtttgagcttgtggtcctcctcaatgccactgtggaatccaccagcgctgtctgccagagccgaacatcttatatcccagaggaaatctactggggttttgagtttgtgcctgtggtatctctctccaaaaatggaaaatatgtggctgatttcagtcagtttgaacagattcggaaaagcccagattgcacattttactgtgcagattctgagaaacagcaactcgaggagaagtacaggcaggaggatcagagggaaagagaactgaggacacttttattacaacagagcaatgtctgatcacaggggcgccatccaggtttaaccctgcaagctgtttccacatcagaactcccttcaaacacaaagattgctgtgaaaacgaaaatgtgtagacgcactctcaaaaactgcacggacatacaaaatcaatcttttcctttgatcttgtggctaaaccagcatttctgtgtttgagagatttcctgttaggtgcttcgtctgaaagtgaactctcataattcaaattgtataaaataaagctacatttctaagagcttggtgtagggcaattggaataatgtcctgttggataaacagacatttagcaatgctgacattaaaaggaaatgtatttctatacaagattattagctgtaatacaagatatttatttaaccaatgaccttatggctgagagttgaattgtggttcagtattcattgatctcactgctttaaaacatgctccttttgttcaagcag

SEQ ID NO: 8 - protein, 375 aa, Human
MDAIHIGMSSTPLVKHTAGAGLKANRPRVMSKSGHSNVRIDKVDGIYLLYLQDLWTTVIDMKWRYKLTLFAATFVMTWFLFGVIYYAIAFIHGDLEPGEPISNHTPCIMKVDSLTGAFLFSLESQTTIGYGVRSITEECPHAIFLLVAQLVITTLIEIFITGTFLAKIARPKKRAETIKFSHCAVITKQNGKLCLVIQVANMRKSLLIQCQLSGKLLQTHVTKEGERILLNQATAKFHVDSSSESPFLILPMTFYHVLDETSPLRDLTPQNLKEKEFELVVLLNATVESTSAVCQSRTSYIPEEIYWGFEFVPVVSLSKNGKYVADFSQFEQIRKSPDCTFYCADSEKQQLEEKYRQEDQRERELRTLLLQQSNV